(19) United States
(12) Patent Application Publication
(10) Pub. No.: US 2026/0327293 A1
(43) Pub. Date: Oct. 22, 2026

(54) INFECTIOUS DISEASE ANTIBODIES AND USES THEREOF

(71) Applicant: Moonshot Antibodies, LLC, Shelburne, VT (US)

(72) Inventors: David N. Krag, Shelburne, VT (US); Girja S. Shukla, South Burlington, VT (US); Stephanie C. Fournier, Colchester, VT (US); Sunny Yujing Sun, South Burlington, VT (US)

(21) Appl. No.: 19/633,053

(22) Filed: Mar. 30, 2026

Related U.S. Application Data
(63) Continuation of application No. 17/918,966, filed on Apr. 13, 2021, now abandoned.
(60) Provisional application No. 63/009,924, filed on Apr. 14, 2020.

(51) Int. Cl. C07K 16/104; A61P 31/14; C12N 5/078

(52) U.S. Cl. CPC C07K 16/104; A61P 31/14; C12N 5/0634; C07K 2317/10; C07K 2317/21

(57) ABSTRACT

Compositions and methods involving effector cells armed with antiviral antibodies are disclosed herein. Such compositions may also include additional antiviral treatments and may be used, for example, to treat subjects having a viral infection (e.g., SARS-COV-2).

### RELATED APPLICATION

This application claims the benefit under 35 U.S.C. § 119(e) of U.S. provisional application No. 63/009,924, filed Apr. 14, 2020, which is incorporated by reference herein in its entirety.

### SEQUENCE LISTING

The instant application contains a Sequence Listing which has been submitted electronically in XML file format and is hereby incorporated by reference in its entirety. Said XML copy, created on Jul. 2, 2026, is named 000229-000202US_SL.xml and is 2,226 bytes in size.

### BACKGROUND OF THE INVENTION

Infectious diseases are an international problem. Caused by microorganisms, such as bacteria, viruses, fungi, or parasites, infectious diseases may spread rapidly through the human population. In some instances, the diseases are mild and can be treated with rest and home remedies; however, others, such as novel viral diseases, may be fatal to the more vulnerable members of the population.

### SUMMARY OF THE INVENTION

The disclosure, in some aspects, provides a method of treating an infectious disease (e.g., viral disease) in a subject, the method comprising administering to a subject having the infectious disease, effector cells comprising antibodies (e.g., two or more antibodies) in an effective amount to treat the infectious disease. By “arming” effector cells with antibodies, lower volumes of antibodies are required for therapeutic effects. In this way, convalescent plasma and other sources of antibodies may be used to treat multiple subjects in need of treatment.

In some embodiments, the antibodies are derived from convalescent plasma. In some embodiments, the convalescent plasma is enriched.

In some embodiments, the effector cells are autologous effector cells. In some embodiments, the effector cells are white blood cells (WBCs). In some embodiments, the effector cells are selected from the group consisting of natural killer (NK) cells, neutrophils, T cells, B cells, monocytes/macrophages, and combinations thereof. In some embodiments, the effector cells are administered to the subject at least three times.

In some embodiments, the infectious disease is selected from the group consisting of: a viral disease, a bacterial disease, a fungal disease, and a parasitic disease. In some embodiments, the viral disease is selected from the group consisting of: coronavirus, HTLV, HTLV-1, HIV/AIDS, human papilloma virus, herpes virus, herpes, genital herpes, viral dysentery, colds, flu, measles, rubella, Chickenpox, mumps, gray myelitis, rabies, mononucleosis, Ebola, respiratory syncytial virus, dengue, yellow fever, Lassa fever, viral meningitis, western Nile fever, parainfluenza, chickenpox, smallpox, dengue hemorrhagic fever, progressive multiple Progressive multifocal leukoencephalopathy, viral gastroenteritis, acute appendicitis, hepatitis A, hepatitis B, chronic hepatitis B, hepatitis C, chronic hepatitis C, hepatitis D, hepatitis E, hepatitis X, simple Herpes, shingles, meningitis, encephalitis, shingles, pneumonia, encephalitis, California serogroup virus, St. Louis encephalitis, Rift Valley fever, Hand, foot & mouth disease, hendravirus, Japanese encephalitis, lymphocytic choroiditis, sudden rash, dorsal parasite virus, SARS, warts, and cerebellar disease. In one embodiment, the virus is a coronavirus. In one embodiment, the coronavirus is SARS-COV-2 (COVID-19).

In some embodiment, the effector cells produce a significant reduction in infectious disease severity relative to administration of effector cells without antibodies. In some embodiments, the effector cells produce a significant reduction in infectious disease severity. In some embodiments, the infectious disease is a viral disease and the reduction in infectious disease severity is a reduction in viral load.

In some embodiments, the effector cells produce a significant increase in survival rate relative to administration of effector cells without antibodies. In some embodiments, the effector cells produce a significant increase in survival rate.

In some embodiments, the effector cells produce a durable immune response relative to administration of effector cells without antibodies. In some embodiments, the durable immune response lasts for at least six months.

In some embodiments, the any one of the methods described herein further comprise administering an anti-infectious disease agent.

The disclosure, in another aspect, provides a composition comprising effector cells comprising two or more anti-infectious disease antibodies and a pharmaceutically acceptable carrier.

In some embodiments, the effector cells and antibodies are derived from different subjects. In some embodiments, the autologous effector cells are selected from the group consisting of natural killer (NK) cells, neutrophils, T cells, B cells, monocytes/macrophages, and combinations thereof.

In some embodiments, the anti-infectious disease antibodies are directed to bacterial antigens, viral antigens, parasitic antigens, or fungal antigens. In some embodiments, the viral antigens are antigens associated with SARS-COV-2 (COVID19).

In some embodiments, the antibodies are derived from convalescent plasma. In some embodiments, the antibodies are monoclonal antibodies. In some embodiments, the antibodies are polyclonal antibodies. In some embodiments, the polyclonal antibodies are human antibodies. In some embodiments, the antibodies are pooled human antibodies.

In another aspect, the disclosure provides a method of treating an infectious disease in a subject, the method comprising: harvesting convalescent plasma from at least one donor; collecting effector cells from the subject; incubating the convalescent plasma and effector cells together, thereby generating armed effector cells; and administering the armed effector cells to the subject in an effective amount to treat the infectious disease.

In some embodiments, the effector cells are selected from the group consisting of natural killer (NK) cells, neutrophils, T cells, B cells, monocytes/macrophages, and combinations thereof. In some embodiments, the effector cells are armed with antibodies directed to bacterial antigens, viral antigens, parasitic antigens, or fungal antigens. In some embodiments, the viral antigens are antigens associated with SARS-CoV-2 (COVID19). In some embodiments, the antibodies are pooled human antibodies.

In some embodiments, any one of the methods provided herein further comprises enriching the convalescent plasma.

In some embodiments, the effector cells are administered to the subject at least three times. In some embodiments, the effector cells are administered daily. In some embodiments, 50 mL of convalescent plasma is used to arm the effector cells.

The disclosure, in another aspect, provides a composition comprising effector cells comprising an anti-infectious disease antibody and a pharmaceutically acceptable carrier, wherein the anti-infectious disease antibody is directed to antigens associated with SARS-COV-2 (COVID19).

Each of the limitations of the invention can encompass various embodiments of the invention. It is, therefore, anticipated that each of the limitations of the invention involving any one element or combinations of elements can be included in each aspect of the invention. This invention is not limited in its application to the details of construction and the arrangement of components set forth in the following description or illustrated in the drawings. The invention is capable of other embodiments and of being practiced or of being carried out in various ways. The details of one or more embodiments of the invention are set forth in the accompanying Detailed Description, Example, Claims, and Figures Other features, objects, and advantages of the invention will be apparent from the description and from the claims.

### BRIEF DESCRIPTION OF DRAWING

The accompanying drawing is not intended to be drawn to scale. In the drawing, each identical or nearly identical component that is illustrated in various figures is represented by a like numeral. For purposes of clarity, not every component may be labeled in every drawing. In the drawings:

FIG. 1 is a schematic illustrating a single treatment described in Example 1.

### DETAILED DESCRIPTION OF THE INVENTION

The present disclosure relates, in one aspect, to the use of effector cells armed with anti-infectious disease antibodies (e.g., antiviral antibodies) for the treatment of subjects having one or more infectious diseases. For example, the subject may have SARS-COV-2, and may be treated with white blood cells (WBCs) armed with low doses of anti-SARS-COV-2 antibodies. By arming effector cells with antibodies, smaller volumes of antibodies are needed, and therefore, the population of patients may benefit by the ability to treat a large number of patients from a limited source of antibodies.

This may provide treatment options early in the course of viral outbreak when reagents are in very short supply. Also, one of the fundamental problems early in an outbreak is the lack of standardization of source antibodies. This is because so many antibodies are needed to treat individual patients that any batch of convalescent plasma will be available for very few people. Without wishing to be bound by theory, it is thought that, by expanding the use of small supplies of reagents using the protocol described herein, multiple patients could be treated from the same limited supply of reagent, permitting a quicker and more widespread application of convalescent plasma. Further, this protocol also describes a method that will allow evaluation of specific antibodies (e.g., anti-SARS-COV-2 antibodies) sooner than otherwise possible. This is because each treatment will require a small quantity of total antibody.

Therefore, the present disclosure relates, in one aspect, to methods of cell therapy using anti-infectious agent antibodies to redirect multiple types of immune effector cells (e.g., white blood cells) to multiple targets (e.g., infected cells). In one embodiment, the disclosure provides methods of cell therapy using antiviral antibodies to redirect multiple types of immune effector cells (e.g., white blood cells) to multiple targets (e.g., virus-infected cells). Prior to delivery, effector cells may be coated ex vivo with antibodies (e.g., a cocktails of antiviral antibodies, as described herein). Without wishing to be bound by theory, it is thought that simultaneously targeting multiple infectious disease-specific antigens may overcome problems associated with the potency of different antibodies. Incubating effector cells ex vivo vastly reduces the amount of antibody required to cause a therapeutic response. Unlike conventional T cell therapy, expansion of effector cells ex vivo is not required.

Further, the protocols described herein may be used to develop a treatment method which can be deployed early in the phase of an infectious disease outbreak (e.g., using convalescent plasma) before other effective modalities become available. The treatments described herein may also improve control over the bioactivity of administered antibodies. For example, control of antibody activity is critical in patients infected with SARS-COV-2, since such patients have lung injury directly related to inflammation. Currently, convalescent plasma is administered in high volumes, such as from 200 ml to 500 ml. These large volumes of antibodies remain present for weeks. If the antibodies cause increasing pulmonary inflammation, this will be difficult or impossible to stop. However, as described herein, each treatment of armed WBCs will deliver about 50 mL of convalescent plasma. The antibodies will be bioactive when bound to the WBCs for 24 hours or less, and when the antibodies are released from the WBCs, systemic levels will be negligible. Thus, there is rapid and automatic cessation of antibody activity when the armed WBCs are no longer armed. Potential worsening of lung pathology by antibodies is mitigated by the very short duration action.

The antibodies (e.g., anti-SARS-COV-2 antibodies), in some embodiments, come from convalescent plasma. The basis for clinical application of convalescent plasma to treat a variety of infections is well grounded in preclinical data. For example, treatment with polyclonal IgG from convalescent plasma of nonhuman primates (NHPs) recovered from filovirus infection has been found to protect other newly infected NHPs, even when administered after clinical manifestations of the disease were already present. [1] Further, plasma from HIV patients prepared as pooled immunoglobulins was found to have better anti-HIV bioactivity than plasma pooled from the general population. [2] Convalescent plasma contains antibodies that target multiple epitopes which may be beneficial. For example, in a controlled analysis with individual hybridoma antibodies that had variable protection against herpes simplex virus type 2, a cocktail of 6 antibodies provided the best protection. [3]

In some embodiments, the convalescent plasma has not been immune-enriched to the infectious antigens. In other embodiments, the convalescent plasma has been enriched to the infectious antigens (e.g., SARS-COV-2 antigens). Without wishing to be bound by theory, it is thought that enriched convalescent plasma provides a high ratio of antibodies against antigens (e.g., SARS-COV-2 antigens) and therefore provides a higher ratio of antibodies on the surface of armed effector cells (e.g., armed WBCs).

Convalescent plasma may be obtained using any method known in the art, such as with apheresis. In some embodiments, the convalescent plasma may be screened for its antibodies' neutralizing activity, e.g., with a plaque reduction neutralization test in combination with an ELISA. In some embodiments, the antibodies may be identified from the convalescent plasma.

Convalescent plasma from a few donors could provide enough antibodies for hundreds of treatments with the methods described herein. Massive efforts are underway to better define anti-SARS-COV-2 antibodies using a wide variety of methods such as SARS-COV-2 antigen immunopurification from convalescent plasma, humanized animals vaccinated with SARS-COV-2 antigen(s), and molecular methods such as individual B cell screening and cloning of the antibody. Any of the antibodies generated with these other methods may be used to arm effector cells, as described herein.

Some aspects of the disclosure relate to anti-infectious agent antibodies, e.g., molecules that bind infectious agents or a fragment thereof. For example, the anti-infectious antibody may be an antiviral antibody, an antibacterial antibody, an anti-fungal antibody, or an anti-parasitic antibody. As used herein, the term “anti-infectious agent antibody” refers to any antibody capable of binding to an infectious microorganism or a fragment thereof. As used herein, the term “antiviral antibody” refers to any antibody capable of binding to a virus (e.g., SARS-COV-2) (direct neutralization). In some instances, the antibody can suppress the bioactivity of the infectious agent, and by extension, its expansion and/or propagation. With respect to antiviral antibodies, mechanisms of antiviral activity of antibodies include direct neutralization of the virus which primarily involves the variable end of the antibody, and antibody interaction with WBCs which primarily involves the Fc end of the antibody. The Fc end of the antibody, described below, engages multiple types of cells of the innate immune system. These cells deploy a wide variety of mechanisms to inhibit viral growth.

An antibody (interchangeably used in plural form) is an immunoglobulin molecule capable of specific binding to a target, such as a carbohydrate, polynucleotide, lipid, polypeptide, etc., through at least one antigen recognition site, located in the variable region of the immunoglobulin molecule. As used herein, the term “antibody” encompasses not only intact (i.e., full-length) polyclonal or monoclonal antibodies, but also antigen-binding fragments thereof (such as Fab, Fab′, F(ab′) 2, Fv), single chain (scFv), mutants thereof, fusion proteins comprising an antibody portion, humanized antibodies, chimeric antibodies, diabodies, nanobodies, linear antibodies, single chain antibodies, multi-specific antibodies (e.g., bispecific antibodies) and any other modified configuration of the immunoglobulin molecule that comprises an antigen recognition site of the required specificity, including glycosylation variants of antibodies, amino acid sequence variants of antibodies, and covalently modified antibodies. An antibody includes an antibody of any class, such as IgD, IgE, IgG, IgA, or IgM (or sub-class thereof), and the antibody need not be of any particular class. Depending on the antibody amino acid sequence of the constant domain of its heavy chains, immunoglobulins can be assigned to different classes. There are five major classes of immunoglobulins: IgA, IgD, IgE, IgG, and IgM, and several of these may be further divided into subclasses (isotypes), e.g., IgG1, IgG2, IgG3, IgG4, IgA1 and IgA2. The heavy-chain constant domains that correspond to the different classes of immunoglobulins are called alpha, delta, epsilon, gamma, and mu, respectively. The subunit structures and three-dimensional configurations of different classes of immunoglobulins are well known.

A typical antibody molecule comprises a heavy chain variable region (V_{H}) and a light chain variable region (V_{L}), which are usually involved in antigen binding. The V_{H} and V_{L} regions can be further subdivided into regions of hypervariability, also known as “complementarity determining regions” (“CDR”), interspersed with regions that are more conserved, which are known as “framework regions” (“FR”). Each V_{H} and V_{L} is typically composed of three CDRs and four FRs, arranged from amino-terminus to carboxy-terminus in the following order: FR1, CDR1, FR2, CDR2, FR3, CDR3, FR4. The extent of the framework region and CDRs can be precisely identified using methodology known in the art, for example, by the Kabat definition, the Chothia definition, the IMGT definition the AbM definition, and/or the contact definition, all of which are well known in the art. See, e.g., Kabat, E. A., et al. (1991) Sequences of Proteins of Immunological Interest, Fifth Edition, U.S. Department of Health and Human Services, NIH Publication No. 91-3242, Chothia et al., (1989) Nature 342:877; Chothia, C. et al. (1987) J. Mol. Biol. 196:901-917, Al-lazikani et al (1997) J. Molec. Biol. 273:927-948; Ye et al., Nucleic Acids Res., 2013, 41: W34-40, and Almagro, J. Mol. Recognit. 17:132-143 (2004). See also hgmp.mrc.ac.uk and bioinf.org.uk/abs).

The antibodies described herein may be full-length antibodies, which contain two heavy chains and two light chains, each including a variable domain and a constant domain. Alternatively, the antibodies can be antigen-binding fragments of a full-length antibody. Examples of binding fragments encompassed within the term “antigen-binding fragment” of a full length antibody include (i) a Fab fragment, a monovalent fragment consisting of the V_{L}, V_{H}, C_{L} and C_{H}1 domains; (ii) a F(ab′)₂ fragment, a bivalent fragment including two Fab fragments linked by a disulfide bridge at the hinge region; (iii) a Fd fragment consisting of the V_{H} and C_{H}1 domains; (iv) a Fv fragment consisting of the V_{L} and V_{H} domains of a single arm of an antibody, (v) a dAb fragment (Ward et al., (1989) Nature 341:544-546), which consists of a V_{H} domain; and (vi) an isolated complementarity determining region (CDR) that retains functionality. Furthermore, although the two domains of the Fv fragment, V_{L} and V_{H}, are coded for by separate genes, they can be joined, using recombinant methods, by a synthetic linker that enables them to be made as a single protein chain in which the V_{L} and V_{H} regions pair to form monovalent molecules known as single chain Fv (scFv). See e.g., Bird et al. (1988) Science 242:423-426; and Huston et al. (1988) Proc. Natl. Acad. Sci. USA 85:5879-5883.

In some embodiments, the anti-infectious disease antibody as described herein can bind and inhibit the viral dissemination of the infectious microorganism by at least 50% (e.g., 60%, 70%, 80%, 90%, 95% or greater). For example, the ability of the infectious agent to multiply and invade surrounding cells may be inhibited by the antibody. The apparent inhibition constant (Ki^{app} or K^{i,app}), which provides a measure of inhibitor potency, is related to the concentration of inhibitor (e.g., antibody) required to reduce enzyme activity and is not dependent on enzyme concentrations. The inhibitory activity of an antibody described herein can be determined by routine methods known in the art.

The Kᵢ,^{app} value of an antibody may be determined by measuring the inhibitory effect of different concentrations of the antibody on the extent of the reaction (e.g., enzyme activity); fitting the change in pseudo-first order rate constant (ν) as a function of inhibitor concentration to the modified Morrison equation (Equation 1) yields an estimate of the apparent Ki value. For a competitive inhibitor, the Ki^{app} can be obtained from the y-intercept extracted from a linear regression analysis of a plot of Kᵢ,^{aPP} versus substrate concentration.

$$v = A \cdot \frac{( [ E ] - [ I ] - {K}_{i}^{app} ) + \sqrt{{( [ E ] - [ I ] - {K}_{i}^{app} )}^{2} + 4 [ E ] \cdot } {K}_{i}^{app}}{2} \qquad ( Equation 1 )$$

Where A is equivalent to νₒ/E, the initial velocity (νₒ) of the enzymatic reaction in the absence of inhibitor (I) divided by the total enzyme concentration (E).

In some embodiments, the antiviral antibody described herein may have a Ki^{app} value of 1000, 900, 800, 700, 600, 500, 400, 300, 200, 100, 50, 40, 30, 20, 19, 18, 17, 16, 15, 14, 13, 12, 11, 10, 9, 8, 7, 6, 5 μM or less for the target infectious agent or infectious agent epitope. In some embodiments, the antibody may have a lower Kᵢ^{app} for a first target (e.g., one epitope of an infectious agent) relative to a second target (e.g., a second epitope of the infectious agent). Differences in Ki^{app} (e.g., for specificity or other comparisons) can be at least 1.5, 2, 3, 4, 5, 10, 15, 20, 37.5, 50, 70, 80, 91, 100, 500, 1000, 10,000 or 105 fold. In some examples, the antibody inhibits a first antigen (e.g., a first protein in a first conformation or mimic thereof) better relative to a second antigen (e.g., the same first protein in a second conformation or mimic thereof; or a second protein). In some embodiments, any of the antibodies may be further affinity matured to reduce the Ki^{app} of the antibody to the target infectious agent or infectious agent epitope thereof.

The antibodies described herein can be murine, rat, rabbit, human, or any other origin (including chimeric or humanized antibodies). Such antibodies are non-naturally occurring, i.e., would not be produced in an animal without human act (e.g., immunizing such an animal with a desired antigen or fragment thereof or isolated from antibody libraries). In some instances, the antibodies are pooled antibodies (e.g., from more than one source). In other instances, the antibodies are from one donor. In one instance, the antibodies are pooled human antibodies. In some embodiments the antibodies are from convalescent plasma.

Any of the antibodies described herein can be either monoclonal or polyclonal. A “monoclonal antibody” refers to a homogenous antibody population and a “polyclonal antibody” refers to a heterogeneous antibody population. These two terms do not limit the source of an antibody or the manner in which it is made.

In one example, the antibody used in the methods described herein is a humanized antibody. Humanized antibodies refer to forms of non-human (e.g., murine) antibodies that are specific chimeric immunoglobulins, immunoglobulin chains, or antigen-binding fragments thereof that contain minimal sequence derived from non-human immunoglobulin. For the most part, humanized antibodies are human immunoglobulins (recipient antibody) in which residues from a CDR of the recipient are replaced by residues from a CDR of a non-human species (donor antibody) such as mouse, rat, or rabbit having the desired specificity, affinity, and capacity. In some instances, Fv framework region (FR) residues of the human immunoglobulin are replaced by corresponding non-human residues. Furthermore, the humanized antibody may comprise residues that are found neither in the recipient antibody nor in the imported CDR or framework sequences, but are included to further refine and optimize antibody performance. In general, the humanized antibody will comprise substantially all of at least one, and typically two, variable domains, in which all or substantially all of the CDR regions correspond to those of a non-human immunoglobulin and all or substantially all of the FR regions are those of a human immunoglobulin consensus sequence. The humanized antibody optimally also will comprise at least a portion of an immunoglobulin constant region or domain (Fc), typically that of a human immunoglobulin. Antibodies may have Fc regions modified as described in WO 99/58572. Other forms of humanized antibodies have one or more CDRs (one, two, three, four, five, or six) which are altered with respect to the original antibody, which are also termed one or more CDRs “derived from” one or more CDRs from the original antibody. Humanized antibodies may also involve affinity maturation.

Methods for constructing humanized antibodies are also well known in the art. See, e.g., Queen et al., Proc. Natl. Acad. Sci. USA, 86:10029-10033 (1989). In one example, variable regions of V_{H} and V_{L} of a parent non-human antibody are subjected to three-dimensional molecular modeling analysis following methods known in the art. Next, framework amino acid residues predicted to be important for the formation of the correct CDR structures are identified using the same molecular modeling analysis. In parallel, human V_{H} and V_{L} chains having amino acid sequences that are homologous to those of the parent non-human antibody are identified from any antibody gene database using the parent V_{H} and V_{L} sequences as search queries. Human V_{H} and V_{L} acceptor genes are then selected.

The CDR regions within the selected human acceptor genes can be replaced with the CDR regions from the parent non-human antibody or functional variants thereof. When necessary, residues within the framework regions of the parent chain that are predicted to be important in interacting with the CDR regions can be used to substitute for the corresponding residues in the human acceptor genes.

In another example, the antibody described herein is a chimeric antibody, which can include a heavy constant region and a light constant region from a human antibody. Chimeric antibodies refer to antibodies having a variable region or part of variable region from a first species and a constant region from a second species. Typically, in these chimeric antibodies, the variable region of both light and heavy chains mimics the variable regions of antibodies derived from one species of mammals (e.g., a non-human mammal such as mouse, rabbit, and rat), while the constant portions are homologous to the sequences in antibodies derived from another mammal such as human. In some embodiments, amino acid modifications can be made in the variable region and/or the constant region. Modifications can include naturally occurring amino acids and non-naturally occurring amino acids. Examples of non-naturally occurring amino acids are modifications that are not isotypic and can be found in U.S. Pat. No. 6,586,207; WO 98/48032; WO 03/073238; US2004-0214988A1; WO 05/35727A2; WO 05/74524A2; J. W. Chin et al., (2002), Journal of the American Chemical Society 124:9026-9027; J. W. Chin, & P. G. Schultz, (2002), ChemBioChem 11:1135-1137; J. W. Chin, et al., (2002), PICAS United States of America 99:11020-11024; and, L. Wang, & P. G. Schultz, (2002), Chem. 1-10, each of which is incorporated by reference herein in its entirety.

In some embodiments, the antiviral antibodies described herein specifically bind to the corresponding target infectious agent or an epitope thereof. An antibody that “specifically binds” to an infectious agent or an epitope is a term well understood in the art. A molecule is said to exhibit “specific binding” if it reacts more frequently, more rapidly, with greater duration and/or with greater affinity with a particular target antigen than it does with alternative targets. An antibody “specifically binds” to a target antigen or epitope if it binds with greater affinity, avidity, more readily, and/or with greater duration than it binds to other substances. For example, an antibody that specifically (or preferentially) binds to an infectious agent or fragment thereof (e.g., epitope) is an antibody that binds this target antigen with greater affinity, avidity, more readily, and/or with greater duration than it binds to other infectious agents or other epitopes in the same infectious agent. It is also understood with this definition that, for example, an antibody that specifically binds to a first target infectious agent may or may not specifically or preferentially bind to a second target infectious agent. As such, “specific binding” or “preferential binding” does not necessarily require (although it can include) exclusive binding. In some examples, an antibody that “specifically binds” to a target antigen or an epitope thereof may not bind to other antigens or other epitopes in the same antigen (i.e., only baseline binding activity can be detected in a conventional method). In some embodiments, the antibodies described herein specifically bind to a selected epitope of an infectious agent. In one embodiment, the antibodies described herein specifically bind to SARS-COV-2 or an epitope thereof.

In some embodiments, an antibody as described herein has a suitable binding affinity for the target antigen (e.g., infectious agent) or epitope(s) thereof. As used herein, “binding affinity” refers to the apparent association constant or K_{A}. The K_{A} is the reciprocal of the dissociation constant (K_{D}). The antibodies described herein may have a binding affinity (K_{D}) of at least 10⁻⁵, 10⁻⁶, 10⁻⁷, 10⁻⁸, 10⁻⁹, 10⁻¹⁰ M, or lower for the target infectious agent or epitope thereof. An increased binding affinity corresponds to a decreased K_{D}. Higher affinity binding of an antibody for a first antigen relative to a second antigen can be indicated by a higher K_{A} (or a smaller numerical value K_{D}) for binding the first antigen than the K_{A} (or numerical value K_{D}) for binding the second antigen. In such cases, the antibody has specificity for the first antigen (e.g., a first protein in a first conformation or mimic thereof) relative to the second antigen (e.g., the same first protein in a second conformation or mimic thereof; or a second protein). In some embodiments, the antibodies described herein have a higher binding affinity (a higher K_{A} or smaller K_{D}) to a specific infectious agent as compared to the binding affinity to a second infectious agent. Differences in binding affinity (e.g., for specificity or other comparisons) can be at least 1.5, 2, 3, 4, 5, 10, 15, 20, 37.5, 50, 70, 80, 91, 100, 500, 1000, 10,000 or 105 fold. In some embodiments, any of the antiviral antibodies may be further affinity matured to increase the binding affinity of the antibody to the target infectious agent or antigenic epitope thereof.

Binding affinity (or binding specificity) can be determined by a variety of methods including equilibrium dialysis, equilibrium binding, gel filtration, ELISA, surface plasmon resonance, or spectroscopy (e.g., using a fluorescence assay). Exemplary conditions for evaluating binding affinity are in HBS-P buffer (10 mM HEPES pH7.4, 150 mM NaCl, 0.005% (v/v) Surfactant P20). These techniques can be used to measure the concentration of bound binding protein as a function of target protein concentration. The concentration of bound binding protein ([Bound]) is generally related to the concentration of free target protein ([Free]) by the following equation:

[Bound]=[Free]/(Kd+[Free])

It is not always necessary to make an exact determination of K_{A}, though, since sometimes it is sufficient to obtain a quantitative measurement of affinity, e.g., determined using a method such as ELISA or FACS analysis, is proportional to K_{A}, and thus can be used for comparisons, such as determining whether a higher affinity is, e.g., 2-fold higher, to obtain a qualitative measurement of affinity, or to obtain an inference of affinity, e.g., by activity in a functional assay, e.g., an in vitro or in vivo assay.

In some embodiments, the antibodies described herein bind to the same epitope as any of the exemplary antibodies described herein or competes against the exemplary antibody from binding to the infectious agent. An “epitope” refers to the site on a target infectious agent that is recognized and bound by an antibody. The site can be entirely composed of amino acid components, entirely composed of chemical modifications of amino acids of the protein (e.g., glycosyl moieties), or composed of combinations thereof. Overlapping epitopes include at least one common amino acid residue. An epitope can be linear, which is typically 6-15 amino acids in length. Alternatively, the epitope can be conformational. The epitope to which an antibody binds can be determined by routine technology, for example, the epitope mapping method (see, e.g., descriptions below). An antibody that binds the same epitope as an exemplary antibody described herein may bind to exactly the same epitope or a substantially overlapping epitope (e.g., containing less than 3 non-overlapping amino acid residue, less than 2 non-overlapping amino acid residues, or only 1 non-overlapping amino acid residue) as the exemplary antibody. Whether two antibodies compete against each other from binding to the cognate antigen can be determined by a competition assay, which is well known in the art.

Also within the scope of the present disclosure are functional variants of any of the exemplary antiviral antibodies as disclosed herein. Such functional variants are substantially similar to the exemplary antibody, both structurally and functionally. A functional variant comprises substantially the same V_{H} and V_{L} CDRs as the exemplary antibody. For example, it may comprise only up to 5 (e.g., 4, 3, 2, or 1) amino acid residue variations in the total CDR regions of the antibody and binds the same epitope of the infectious agent with substantially similar affinity (e.g., having a K_{D} value in the same order). Alternatively or in addition, the amino acid residue variations are conservative amino acid residue substitutions. As used herein, a “conservative amino acid substitution” refers to an amino acid substitution that does not alter the relative charge or size characteristics of the protein in which the amino acid substitution is made. Variants can be prepared according to methods for altering polypeptide sequence known to one of ordinary skill in the art such as are found in references which compile such methods, e.g. Molecular Cloning: A Laboratory Manual, J. Sambrook, et al., eds., Second Edition, Cold Spring Harbor Laboratory Press, Cold Spring Harbor, New York, 1989, or Current Protocols in Molecular Biology, F. M. Ausubel, et al., eds., John Wiley & Sons, Inc., New York. Conservative substitutions of amino acids include substitutions made amongst amino acids within the following groups: (a) M, I, L, V; (b) F, Y, W; (c) K, R, H; (d) A, G; (e) S, T; (f) Q, N; and (g) E, D.

In some embodiments, the antiviral antibody may comprise heavy chain CDRs that share at least 80% (e.g., 85%, 90%, 95%, or 98%) sequence identity, individually or collectively, with the V_{H} CDRs of an antibody described herein. Alternatively or in addition, the antiviral antibody may comprise light chain CDRs that share at least 80% (e.g., 85%, 90%, 95%, or 98%) sequence identity, individually or collectively, with the V_{L} CDRs as an antibody described herein.

The “percent identity” of two amino acid sequences is determined using the algorithm of Karlin and Altschul Proc. Natl. Acad. Sci. USA 87:2264-68, 1990, modified as in Karlin and Altschul Proc. Natl. Acad. Sci. USA 90:5873-77, 1993. Such an algorithm is incorporated into the NBLAST and XBLAST programs (version 2.0) of Altschul, et al. J. Mol. Biol. 215:403-10, 1990. BLAST protein searches can be performed with the XBLAST program, score=50, wordlength=3 to obtain amino acid sequences homologous to the protein molecules of interest. Where gaps exist between two sequences, Gapped BLAST can be utilized as described in Altschul et al., Nucleic Acids Res. 25(17):3389-3402, 1997. When utilizing BLAST and Gapped BLAST programs, the default parameters of the respective programs (e.g., XBLAST and NBLAST) can be used.

In some embodiments, the heavy chain of any of the antibodies as described herein may further comprise a heavy chain constant region (CH) or a portion thereof (e.g., CH1, CH2, CH3, or a combination thereof). The heavy chain constant region can of any suitable origin, e.g., human, mouse, rat, or rabbit. In one specific example, the heavy chain constant region is from a human IgG (a gamma heavy chain) of any IgG subfamily as described herein. In one example, the constant region is from human IgG4, an exemplary amino acid sequence of which is provided below (SEQ ID NO: 1):

| | | | | | |
|---|---|---|---|---|---|
| ASTKGPSVEP | LAPCSRSTSE | STAALGCLVK | DYFPEPVTVS | WNSGALTSGV | HTFPAVLQSS |
| GLYSLSSVVT | VPSSSLGTKT | YTCNVDHKPS | NTKVDKRVES | KYGPPCPSCP | APEFLGGPSV |
| FLFPPKPKDT | LMISRTPEVT | CVVVDVSQED | PEVQFNWYVD | GVEVHNAKTK | PREEQFNSTY |
| RVVSVLTVLH | QDWLNGKEYK | CKVSNKGLPS | SIEKTISKAK | GQPREPQVYT | LPPSQEEMTK |
| NQVSLTCLVK | GFYPSDIAVE | WESNGQPENN | YKTTPPVLDS | DGSFFLYSRL | TVDKSRWQEG |
| NVFSCSVMHE | ALHNHYTQKS | LSLSLGK | | | |

In some embodiments, the antibody comprises the heavy chain constant region of SEQ ID NO: 1, or a variant thereof, which may contain an S/P substitution at the position as indicated (boldfaced and underlined). Alternatively, the heavy chain constant region of the antibodies described herein may comprise a single domain (e.g., CH1, CH2, or CH3) or a combination of any of the single domains, of a constant region (e.g., SEQ ID NO: 1).

When needed, the antibody as described herein may comprise a modified constant region. For example, it may comprise a modified constant region that is immunologically inert, e.g., does not trigger complement mediated lysis, or does not stimulate antibody-dependent cell mediated cytotoxicity (ADCC). ADCC activity can be assessed using methods disclosed in U.S. Pat. No. 5,500,362. In other embodiments, the constant region is modified as described in Eur. J. Immunol. (1999) 29:2613-2624; PCT Application No. PCT/GB99/01441; and/or UK Patent Application No. 9809951.8.

Any of the antibodies described herein may comprise a light chain that further comprises a light chain constant region, which can be any CL known in the art. In some examples, the CL is a kappa light chain. In other examples, the CL is a lambda light chain.

Antibody heavy and light chain constant regions are well known in the art, e.g., those provided in the IMGT database (www.imgt.org) or at www.vbase2.org/vbstat.php., both of which are incorporated by reference herein.

Antibodies capable of binding to the target antigens as described herein may be isolated from a suitable antibody library via routine practice. Antibody libraries, which contain a plurality of antibody components, can be used to identify antibodies that bind to a specific target infectious agent following routine selection processes as known in the art. In the selection process, an antibody library can be probed with the target antigen or a fragment thereof and members of the library that are capable of binding to the target antigen can be isolated, typically by retention on a support. Such screening process may be performed by multiple rounds (e.g., including both positive and negative selections) to enrich the pool of antibodies capable of binding to the target antigen. Individual clones of the enriched pool can then be isolated and further characterized to identify those having desired binding activity and biological activity. Sequences of the heavy chain and light chain variable domains can also be determined via conventional methodology.

There are a number of routine methods known in the art to identify and isolate antibodies capable of binding to the target infectious agents described herein, including phage display, yeast display, ribosomal display, or mammalian display technology.

As an example, phage displays typically use a covalent linkage to bind the protein (e.g., antibody) component to a bacteriophage coat protein. The linkage results from translation of a nucleic acid encoding the antibody component fused to the coat protein. The linkage can include a flexible peptide linker, a protease site, or an amino acid incorporated as a result of suppression of a stop codon. Phage display is described, for example, in U.S. Pat. No. 5,223,409; Smith (1985) Science 228:1315-1317; WO 92/18619; WO 91/17271; WO 92/20791; WO 92/15679; WO 93/01288; WO 92/01047; WO 92/09690; WO 90/02809; de Haard et al. (1999) J. Biol. Chem 274:18218-30; Hoogenboom et al. (1998) Immunotechnology 4:1-20; Hoogenboom et al. (2000) Immunol Today 2:371-8 and Hoet et al. (2005) Nat Biotechnol. 23(3)344-8. Bacteriophage displaying the protein component can be grown and harvested using standard phage preparatory methods, e.g. PEG precipitation from growth media. After selection of individual display phages, the nucleic acid encoding the selected protein components can be isolated from cells infected with the selected phages or from the phage themselves, after amplification. Individual colonies or plaques can be selected, and then the nucleic acid may be isolated and sequenced.

Other display formats include cell-based display (see, e.g., WO 03/029456), protein-nucleic acid fusions (see, e.g., U.S. Pat. No. 6,207,446), ribosome display (See, e.g., Mattheakis et al. (1994) Proc. Natl. Acad. Sci. USA 91:9022 and Hanes et al. (2000) Nat Biotechnol. 18:1287-92; Hanes et al. (2000) Methods Enzymol. 328:404-30; and Schaffitzel et al. (1999) J Immunol Methods. 231(1-2):119-35), and E. coli periplasmic display (J Immunol Methods. 2005 Nov. 22; PMID: 16337958).

After display library members are isolated for binding to the target antigen, each isolated library member can be also tested for its ability to bind to a non-target molecule to evaluate its binding specificity. Examples of non-target molecules include streptavidin on magnetic beads, blocking agents such as bovine serum albumin, non-fat bovine milk, soy protein, any capturing or target immobilizing monoclonal antibody, or non-transfected cells which do not express the target. A high-throughput ELISA screen can be used to obtain the data, for example. The ELISA screen can also be used to obtain quantitative data for binding of each library member to the target as well as for cross species reactivity to related targets or subunits of the target antigen and also under different condition such as pH 6 or pH 7.5. The non-target and target binding data are compared (e.g., using a computer and software) to identify library members that specifically bind to the target.

After selecting candidate library members that bind to a target, each candidate library member can be further analyzed, e.g., to further characterize its binding properties for the target, e.g., a specific infectious agent. Each candidate library member can be subjected to one or more secondary screening assays. The assay can be for a binding property, a catalytic property, an inhibitory property, a physiological property (e.g., cytotoxicity, renal clearance, immunogenicity), a structural property (e.g., stability, conformation, oligomerization state) or another functional property. The same assay can be used repeatedly, but with varying conditions, e.g., to determine pH, ionic, or thermal sensitivities.

As appropriate, the assays can use a display library member directly, a recombinant polypeptide produced from the nucleic acid encoding the selected polypeptide, or a synthetic peptide synthesized based on the sequence of the selected polypeptide. In the case of selected Fabs, the Fabs can be evaluated or can be modified and produced as intact IgG proteins. Exemplary assays for binding properties are described below.

Binding proteins can also be evaluated using an ELISA assay. For example, each protein is contacted to a microtiter plate whose bottom surface has been coated with the target, e.g., a limiting amount of the target. The plate is washed with buffer to remove non-specifically bound polypeptides. Then the amount of the binding protein bound to the target on the plate is determined by probing the plate with an antibody that can recognize the binding protein, e.g., a tag or constant portion of the binding protein. The antibody is linked to a detection system (e.g., an enzyme such as alkaline phosphatase or horseradish peroxidase (HRP) which produces a colorimetric product when appropriate substrates are provided, chemiluminescent substrates, or fluorescent substrates).

Alternatively, the ability of a binding protein described herein to bind a target antigen can be analyzed using a homogenous assay, i.e., after all components of the assay are added, additional fluid manipulations are not required. For example, fluorescence resonance energy transfer (FRET) can be used as a homogenous assay (see, for example, Lakowicz et al., U.S. Pat. No. 5,631,169; Stavrianopoulos, et al., U.S. U.S. Pat. No. 4,868,103). A fluorophore label on the first molecule (e.g., the molecule identified in the fraction) is selected such that its emitted fluorescent energy can be absorbed by a fluorescent label on a second molecule (e.g., the target) if the second molecule is in proximity to the first molecule. The fluorescent label on the second molecule fluoresces when it absorbs to the transferred energy. Since the efficiency of energy transfer between the labels is related to the distance separating the molecules, the spatial relationship between the molecules can be assessed. In a situation in which binding occurs between the molecules, the fluorescent emission of the ‘acceptor’ molecule label in the assay should be maximal. A binding event that is configured for monitoring by FRET can be conveniently measured through standard fluorometric detection means, e.g., using a fluorimeter. By titrating the amount of the first or second binding molecule, a binding curve can be generated to estimate the equilibrium binding constant.

Surface plasmon resonance (SPR) can be used to analyze the interaction of a binding protein and a target antigen. SPR or Biomolecular Interaction Analysis (BIA) detects biospecific interactions in real time, without labeling any of the interactants. Changes in the mass at the binding surface (indicative of a binding event) of the BIA chip result in alterations of the refractive index of light near the surface (the optical phenomenon of SPR). The changes in the refractivity generate a detectable signal, which are measured as an indication of real-time reactions between biological molecules. Methods for using SPR are described, for example, in U.S. Pat. No. 5,641,640; Raether, 1988, Surface Plasmons Springer Verlag; Sjolander and Urbaniczky, 1991, Anal. Chem. 63:2338-2345; Szabo et al., 1995, Curr. Opin. Struct. Biol. 5:699-705 and on-line resources provide by BIAcore International AB (Uppsala, Sweden).

Information from SPR can be used to provide an accurate and quantitative measure of the equilibrium dissociation constant (K_{D}), and kinetic parameters, including Kₒₙ and K_{off}, for the binding of a binding protein to a target. Such data can be used to compare different biomolecules. For example, selected proteins from an expression library can be compared to identify proteins that have high affinity for the target or that have a slow K_{off}. This information can also be used to develop structure-activity relationships (SAR). For example, the kinetic and equilibrium binding parameters of matured versions of a parent protein can be compared to the parameters of the parent protein. Variant amino acids at given positions can be identified that correlate with particular binding parameters, e.g., high affinity and slow K_{off}. This information can be combined with structural modeling (e.g., using homology modeling, energy minimization, or structure determination by x-ray crystallography or NMR). As a result, an understanding of the physical interaction between the protein and its target can be formulated and used to guide other design processes.

As a further example, cellular assays may be used. Binding proteins can be screened for ability to bind to cells which transiently or stably express and display the target of interest on the cell surface. For example, a target infectious agent's binding protein or proteins can be fluorescently labeled and binding to the infectious agent in the presence or absence of antagonistic antibody can be detected by a change in fluorescence intensity using flow cytometry e.g., a FACS machine.

Antigen-binding fragments of an intact antibody (full-length antibody) can be prepared via routine methods. For example, F(ab′)2 fragments can be produced by pepsin digestion of an antibody molecule, and Fab fragments that can be generated by reducing the disulfide bridges of F(ab′)2 fragments.

Genetically engineered antibodies, such as humanized antibodies, chimeric antibodies, single-chain antibodies, and bi-specific antibodies, can be produced via, e.g., conventional recombinant technology. In one example, DNA encoding a monoclonal antibodies specific to a target antigen can be readily isolated and sequenced using conventional procedures (e.g., by using oligonucleotide probes that are capable of binding specifically to genes encoding the heavy and light chains of the monoclonal antibodies). Once isolated, the DNA may be placed into one or more expression vectors, which are then transfected into host cells such as HEK293 cells, E. coli cells, simian COS cells, Chinese hamster ovary (CHO) cells, or myeloma cells that do not otherwise produce immunoglobulin protein, to obtain the synthesis of monoclonal antibodies in the recombinant host cells. See, e.g., PCT Publication No. WO 87/04462. The DNA can then be modified, for example, by substituting the coding sequence for human heavy and light chain constant domains in place of the homologous murine sequences, Morrison et al., (1984) Proc. Nat. Acad. Sci. 81:6851, or by covalently joining to the immunoglobulin coding sequence all or part of the coding sequence for a non-immunoglobulin polypeptide. In that manner, genetically engineered antibodies, such as “chimeric” or “hybrid” antibodies; can be prepared that have the binding specificity of a target antigen.

Techniques developed for the production of “chimeric antibodies” are well known in the art. See, e.g., Morrison et al. (1984) Proc. Natl. Acad. Sci. USA 81, 6851; Neuberger et al. (1984) Nature 312, 604; and Takeda et al. (1984) Nature 314:452.

Methods for constructing humanized antibodies are also well known in the art. See, e.g., Queen et al., Proc. Natl. Acad. Sci. USA, 86:10029-10033 (1989). In one example, variable regions of V_{H} and V_{L} of a parent non-human antibody are subjected to three-dimensional molecular modeling analysis following methods known in the art. Next, framework amino acid residues predicted to be important for the formation of the correct CDR structures are identified using the same molecular modeling analysis. In parallel, human V_{H} and V_{L} chains having amino acid sequences that are homologous to those of the parent non-human antibody are identified from any antibody gene database using the parent V_{H} and V_{L} sequences as search queries. Human V_{H} and V_{L} acceptor genes are then selected.

The CDR regions within the selected human acceptor genes can be replaced with the CDR regions from the parent non-human antibody or functional variants thereof. When necessary, residues within the framework regions of the parent chain that are predicted to be important in interacting with the CDR regions (see above description) can be used to substitute for the corresponding residues in the human acceptor genes.

A single-chain antibody can be prepared via recombinant technology by linking a nucleotide sequence coding for a heavy chain variable region and a nucleotide sequence coding for a light chain variable region. Preferably, a flexible linker is incorporated between the two variable regions. Alternatively, techniques described for the production of single chain antibodies (U.S. Pat. Nos. 4,946,778 and 4,704,692) can be adapted to produce a phage or yeast scFv library and scFv clones specific to a target infectious agent can be identified from the library following routine procedures. Positive clones can be subjected to further screening to identify those that inhibit the infectious agent's bioactivity (e.g., dissemination, propagation).

Antibodies obtained following a method known in the art and described herein can be characterized using methods well known in the art. For example, one method is to identify the epitope to which the antigen binds, or “epitope mapping.” There are many methods known in the art for mapping and characterizing the location of epitopes on proteins, including solving the crystal structure of an antibody-antigen complex, competition assays, gene fragment expression assays, and synthetic peptide-based assays, as described, for example, in Chapter 11 of Harlow and Lane, Using Antibodies, a Laboratory Manual, Cold Spring Harbor Laboratory Press, Cold Spring Harbor, N.Y., 1999. In an additional example, epitope mapping can be used to determine the sequence, to which an antibody binds. The epitope can be a linear epitope, i.e., contained in a single stretch of amino acids, or a conformational epitope formed by a three-dimensional interaction of amino acids that may not necessarily be contained in a single stretch (primary structure linear sequence). Peptides of varying lengths (e.g., at least 4-6 amino acids long, in some embodiments, 11 amino acids long) can be isolated or synthesized (e.g., recombinantly) and used for binding assays with an antibody. In another example, the epitope to which the antibody binds can be determined in a systematic screening by using overlapping peptides derived from the target antigen sequence and determining binding by the antibody. According to the gene fragment expression assays, the open reading frame encoding the target infectious agent is fragmented either randomly or by specific genetic constructions and the reactivity of the expressed fragments of the antigen with the antibody to be tested is determined. The gene fragments may, for example, be produced by PCR and then transcribed and translated into protein in vitro, in the presence of radioactive amino acids. The binding of the antibody to the radioactively labeled infectious agent fragments is then determined by immunoprecipitation and gel electrophoresis. Certain epitopes can also be identified by using large libraries of random peptide sequences displayed on the surface of phage particles (phage libraries). Alternatively, a defined library of overlapping peptide fragments can be tested for binding to the test antibody in simple binding assays. In an additional example, mutagenesis of an antigen binding domain, domain swapping experiments and alanine scanning mutagenesis can be performed to identify residues required, sufficient, and/or necessary for epitope binding. For example, domain swapping experiments can be performed using a mutant of a target antigen in which various fragments of the infectious agent polypeptide have been replaced (swapped) with sequences from a closely related, but antigenically distinct protein. By assessing binding of the antibody to the mutant infectious agent, the importance of the particular antigen fragment to antibody binding can be assessed.

Alternatively, competition assays can be performed using other antibodies known to bind to the same antigen to determine whether an antibody binds to the same epitope as the other antibodies. Competition assays are well known to those of skill in the art.

In some examples, an antibody is prepared by recombinant technology as exemplified below.

Nucleic acids encoding the heavy and light chain of an antiviral antibody as described herein can be cloned into one expression vector, each nucleotide sequence being in operable linkage to a suitable promoter. In one example, each of the nucleotide sequences encoding the heavy chain and light chain is in operable linkage to a distinct prompter. Alternatively, the nucleotide sequences encoding the heavy chain and the light chain can be in operable linkage with a single promoter, such that both heavy and light chains are expressed from the same promoter. When necessary, an internal ribosomal entry site (IRES) can be inserted between the heavy chain and light chain encoding sequences.

In some examples, the nucleotide sequences encoding the two chains of the antibody are cloned into two vectors, which can be introduced into the same or different cells. When the two chains are expressed in different cells, each of them can be isolated from the host cells expressing such and the isolated heavy chains and light chains can be mixed and incubated under suitable conditions allowing for the formation of the antibody.

Generally, a nucleic acid sequence encoding one or all chains of an antibody can be cloned into a suitable expression vector in operable linkage with a suitable promoter using methods known in the art. For example, the nucleotide sequence and vector can be contacted, under suitable conditions, with a restriction enzyme to create complementary ends on each molecule that can pair with each other and be joined together with a ligase. Alternatively, synthetic nucleic acid linkers can be ligated to the termini of a gene. These synthetic linkers contain nucleic acid sequences that correspond to a particular restriction site in the vector. The selection of expression vectors/promoter would depend on the type of host cells for use in producing the antibodies.

A variety of promoters can be used for expression of the antibodies described herein, including, but not limited to, cytomegalovirus (CMV) intermediate early promoter, a viral LTR such as the Rous sarcoma virus LTR, HIV-LTR, HTLV-1 LTR, the simian virus 40 (SV40) early promoter, E. coli lac UV5 promoter, and the herpes simplex tk virus promoter.

Regulatable promoters can also be used. Such regulatable promoters include those using the lac repressor from E. coli as a transcription modulator to regulate transcription from lac operator-bearing mammalian cell promoters [Brown, M. et al., Cell, 49:603-612 (1987)], those using the tetracycline repressor (tetR) [Gossen, M., and Bujard, H., Proc. Natl. Acad. Sci. USA 89:5547-5551 (1992); Yao, F. et al., Human Gene Therapy, 9:1939-1950 (1998); Shockelt, P., et al., Proc. Natl. Acad. Sci. USA, 92:6522-6526 (1995)]. Other systems include FK506 dimer, VP16 or p65 using astradiol, RU486, diphenol murislerone, or rapamycin. Inducible systems are available from Invitrogen, Clontech and Ariad.

Regulatable promoters that include a repressor with the operon can be used. In one embodiment, the lac repressor from E. coli can function as a transcriptional modulator to regulate transcription from lac operator-bearing mammalian cell promoters [M. Brown et al., Cell, 49:603-612 (1987)]; Gossen and Bujard (1992); [M. Gossen et al., Natl. Acad. Sci. USA, 89:5547-5551 (1992)] combined the tetracycline repressor (tetR) with the transcription activator (VP 16) to create a tetR-mammalian cell transcription activator fusion protein, tTa (tetR-VP 16), with the tetO-bearing minimal promoter derived from the human cytomegalovirus (hCMV) major immediate-early promoter to create a tetR-tet operator system to control gene expression in mammalian cells. In one embodiment, a tetracycline inducible switch is used. The tetracycline repressor (tetR) alone, rather than the tetR-mammalian cell transcription factor fusion derivatives can function as potent trans-modulator to regulate gene expression in mammalian cells when the tetracycline operator is properly positioned downstream for the TATA element of the CMVIE promoter (Yao et al., Human Gene Therapy, 10(16):1392-1399 (2003)). One particular advantage of this tetracycline inducible switch is that it does not require the use of a tetracycline repressor-mammalian cells transactivator or repressor fusion protein, which in some instances can be toxic to cells (Gossen et al., Natl. Acad. Sci. USA, 89:5547-5551 (1992); Shockett et al., Proc. Natl. Acad. Sci. USA, 92:6522-6526 (1995)), to achieve its regulatable effects.

Additionally, the vector can contain, for example, some or all of the following: a selectable marker gene, such as the neomycin gene for selection of stable or transient transfectants in mammalian cells; enhancer/promoter sequences from the immediate early gene of human CMV for high levels of transcription; transcription termination and RNA processing signals from SV40 for mRNA stability; SV40 polyoma origins of replication and ColE1 for proper episomal replication; internal ribosome binding sites (IRESes), versatile multiple cloning sites; and T7 and SP6 RNA promoters for in vitro transcription of sense and antisense RNA. Suitable vectors and methods for producing vectors containing transgenes are well known and available in the art.

Examples of polyadenylation signals useful to practice the methods described herein include, but are not limited to, human collagen I polyadenylation signal, human collagen II polyadenylation signal, and SV40 polyadenylation signal.

One or more vectors (e.g., expression vectors) comprising nucleic acids encoding any of the antibodies may be introduced into suitable host cells for producing the antibodies. The host cells can be cultured under suitable conditions for expression of the antibody or any polypeptide chain thereof. Such antibodies or polypeptide chains thereof can be recovered by the cultured cells (e.g., from the cells or the culture supernatant) via a conventional method, e.g., affinity purification. If necessary, polypeptide chains of the antibody can be incubated under suitable conditions for a suitable period of time allowing for production of the antibody.

In some embodiments, methods for preparing an antibody described herein involve a recombinant expression vector that encodes both the heavy chain and the light chain of an antiviral antibody, as also described herein. The recombinant expression vector can be introduced into a suitable host cell (e.g., a dhfr-CHO cell) by a conventional method, e.g., calcium phosphate-mediated transfection. Positive transformant host cells can be selected and cultured under suitable conditions allowing for the expression of the two polypeptide chains that form the antibody, which can be recovered from the cells or from the culture medium. When necessary, the two chains recovered from the host cells can be incubated under suitable conditions allowing for the formation of the antibody.

In one example, two recombinant expression vectors are provided, one encoding the heavy chain of the antiviral antibody and the other encoding the light chain of the antiviral antibody. Both of the two recombinant expression vectors can be introduced into a suitable host cell (e.g., dhfr-CHO cell) by a conventional method, e.g., calcium phosphate-mediated transfection. Alternatively, each of the expression vectors can be introduced into a suitable host cell. Positive transformants can be selected and cultured under suitable conditions allowing for the expression of the polypeptide chains of the antibody. When the two expression vectors are introduced into the same host cells, the antibody produced therein can be recovered from the host cells or from the culture medium. If necessary, the polypeptide chains can be recovered from the host cells or from the culture medium and then incubated under suitable conditions allowing for formation of the antibody. When the two expression vectors are introduced into different host cells, each of them can be recovered from the corresponding host cells or from the corresponding culture media. The two polypeptide chains can then be incubated under suitable conditions for formation of the antibody.

Standard molecular biology techniques are used to prepare the recombinant expression vector, transfect the host cells, select for transformants, culture the host cells and recovery of the antibodies from the culture medium. For example, some antibodies can be isolated by affinity chromatography with a Protein A or Protein G coupled matrix.

Any of the nucleic acids encoding the heavy chain, the light chain, or both of an antiviral antibody as described herein, vectors (e.g., expression vectors) containing such, and host cells comprising the vectors are within the scope of the present disclosure.

Antibodies thus prepared can be characterized using methods known in the art, whereby reduction, amelioration, or neutralization of tumor (e.g., tumor cell) biological activity is detected and/or measured. For example, an ELISA-type assay may be suitable for qualitative or quantitative measurement of infectious agent bioactivity neutralization.

The present disclosure provides pharmaceutical compositions comprising antibodies described herein (e.g., from convalescent plasma) and uses of such for neutralizing an infectious agent's bioactivity. For example, the antibodies and antigen-binding antibody fragments thereof described herein may be used to treat an infectious disease in a subject. As the antibodies bind the infectious agent with high specificity, they may be used to treat a subject having the infectious disease.

As described herein, effector cells may be coated with the antibodies ex vivo, which reduces the amount of antibody required to treat an infectious disease. Previous studies have demonstrated multiple types of effector cells derived from spleen, peripheral blood, marrow, and peritoneum can mediate cytotoxicity. For example, serum from Guinea pigs immunized with chicken red blood cells was shown to bind firmly to monocyte-macrophages, non-phagocytic lymphocytes, and neutrophils. Binding of the serum rendered the different effector cells cytotoxic to chicken red blood cells. Effector cells derived from human blood demonstrated a range of cytotoxic activity when armed with rabbit antibodies. Non-lymphocytes increased the initial rate of cytotoxicity but purified lymphocytes appeared to have more complete cytotoxicity over time that polymorphonuclear leukocytes and monocytes. Neutrophils, directed by antibodies, have been shown to attack target cells by a unique method of trogocytosis. Using only one cell type as is done with T cell therapy, does not take full advantage of multiple mechanisms of tumor cell destruction by different effector cells.

Despite the fact that multiple effector cell types can participate in antibody-mediated cytotoxicity, the ability to grow T cells has factored heavily in the choice of cell type for most trials. The method described herein avoids the complexity and major expense related to growing T cells ex vivo or genetically modifying T cells. Multiple types of effector cells can be armed since they are readily available from peripheral blood and do not require ex vivo growth expansion.

White blood cells (WBCs) are known to have antiviral properties and deploy a wide variety of mechanisms to inhibit viral growth. “Antibody-dependent cell-mediated viral inhibition” (ADCVI), as used herein, as a general term for all antiviral WBC activities mediated by antibodies. [19] An example of ADCVI is the uptake and removal of virus from the blood for viral inactivation and processing of viral particles to facilitate an immune response. A key mechanism of ADCVI is the destruction of virally infected cells.

Visualization of antibody mediated lymphocyte toxicity of virally infected cells was shown by electron microscopic images. [20] The WBCs attach to abundant viral antigens embedded within the infected target cell membrane. [3] Cell surface viral proteins of SARS-associated coronavirus have been shown to be expressed and transported to the plasma membrane in tissue cells of infected patients. [21] further, electron microscopy shows that IgG from immune plasma reacts with the entire surface of cells infected by foot-and-mouth disease virus. [22] Measles virus-coded proteins are able to move within the plane of the infected cell membrane. [23] These data highlight the abundance and variety of viral particles in the infected cell membrane. [24]

This also further highlights the importance of targeting multiple epitopes to achieve dense attachment of antibodies to the infected cell surface. An immune response to complex targets such as viruses results in antibodies to multiple epitopes. Polyclonal antibodies from immune plasma may be the best method to achieve reliable binding of antibodies to the infected cell surface which displays multiple viral epitopes. [22]

Physical cell-to-cell contact allows WBC killing of the infected cell by processes such as phagocytosis, trogocytosis or target membrane destruction. Contact of the WBC with the virally infected cell is critical. This is mediated by antibodies that form a bridge between the virally infected cell and the WBC. When a threshold of bridging antibodies between the infected cell and the WBC occurs, the WBC will destroy the infected cell. [25] From the perspective of the WBC, there only needs to be sufficient antibodies forming a bridge to the infected cell. The specific viral epitope targeted is of lesser importance.

With direct inhibition of viral function by antibodies, the type and function of the antigenic target is critical. However, for WBCs, achieving threshold binding to the infected cell is the most critical factor. Multiple studies have demonstrated that WBC activation to kill an infected cell is independent of the functional attributes of the antibody-viral interaction. [3, 26, 27] Thus, both neutralizing antibodies and non-neutralizing antibodies can mediate WBC destruction of infected cells. [26] Further evidence shows that monoclonal antibodies reactive with infected cells but not reactive with the intact virus could promote recovery from herpes simplex virus infection. [28]

Convalescent plasma contains abundant antibodies to a wide variety of different viral particles. This, in some embodiments, is valuable for WBC-mediated destruction of infected cells, as the antibody (or antibodies) needs only to attach the infected cell to the WBC. Studies have revealed the existence of a large proportion of clonotypes that recognize the viral antigens only as they are presented in the context of the infected cell, therefore making convalescent plasma a good source of a variety of effective antibodies. [29]

ADCVI is highly clinically relevant. Subjects who succumbed to complications of H7N9 infection demonstrated reduced HA-specific Fc receptor-binding antibodies prior to death compared with those who survived. [30] Further, preclinical models support the biologic action of WBCs. For example, in a model of high dose viral challenge, protection was not achieved by antibody alone, but was by antibody plus human mononuclear cells. [31]

As noted above, preclinical data has shown that WBCs destroy target cells whether the antibodies were first bound to the target cell (via the variable end of the antibody) or first bound to the WBCs (via the Fc portion of the antibody). It has been recently reported that arming white blood cells with a cocktail of antibodies could inhibit tumor at a thousand fold lower dose than the same antibodies given systemically. [37]

A component of this variation in dose effectiveness is that WBCs can be armed in vitro by a high concentration, but low amount, of antibodies. Also, all competitive antibodies contained in blood are rinsed away, thereby giving the loading antibodies the opportunity to bind WBCs in vitro without competition. This is in contrast to when antibodies are injected systemically, as they are instantly diluted. In that dilute state, circulating WBCs are exposed to relatively low concentrations of the administered antibody. In the circulation, there is also competitive binding to Fc receptors by endogenous circulating antibodies.

Therefore, the antibodies described herein may be coated onto effector cells (e.g., WBCs). The resulting effector cells are referred to as “armed effector cells.” Armed effector cells may comprise any number of antibodies, such as 1, 2, 3, 4, 5, 6, 7, 8, 9, 10, 11, 12, 13, 14, 15, 16, 17, 18, 19, 20, or more antibodies. In some embodiments, the antibodies are from convalescent plasma. Examples of effector cells include, but are not limited to white blood cells (leukocytes), such as natural killer (NK) cells, neutrophils, T cells, B cells, and monocytes/macrophages. In some embodiments, a single type of effector cell is used. In other embodiments, a combination of effector cells is used. The effector cells may be coated (“armed”) with the antibodies using any method known in the art. For example, the effector cells may be incubated on ice with convalescent plasma (or cocktail of antibodies, such as antiviral antibodies).

The disclosure, in some aspects, provides a composition effector cells comprising antibodies and a pharmaceutically acceptable carrier (excipient). In some embodiments, the composition comprises two or more antibodies, for instance, 2, 3, 4, 5, 6, 7, 8, 9, 10, 11, 12, 13, 14, 15, 16, 17, 18, 19, 20, or more antibodies. In some embodiments, the two or more antibodies are present in the composition in equal concentrations. In other embodiments, the two or more antibodies are not present in the composition in equal concentrations. In some embodiments, the at least two antibodies are all directed to the same infectious agent. In some embodiments, the at least two antibodies are directed to different epitopes of the same infectious agent. In other embodiments, the at least two antiviral antibodies are not all directed to the same infectious agent. In another embodiment, the at least two antiviral antibodies are all directed to different infectious agent.

In other aspects, the disclosure provides a further therapeutically effective non-antibody anti-infectious agent. As used herein, the term “therapeutically effective” means any agent having anti-infectious agent activity, especially an agent approved for commercial use as an anti-infectious agent and for use in treating and/or preventing infectious diseases in animals, especially in humans.

Provided herein, in some aspects, is a method of treating an infectious disease, the method comprising administering to a subject having the infectious disease, armed effector cells, in an effective amount to treat the infectious disease, wherein the armed effector cells are armed with antibodies from convalescent plasma. In some embodiments, the armed effector cells and a further anti-infectious agent treatment are administered simultaneously. In other embodiments, the armed effector cells are administered to the subject prior to administration of the further anti-infectious agent treatment.

In an additional aspect, the disclosure provides a method of treating a viral disease (e.g., SARS-COV-2), the method comprising administering to a subject having the viral disease, effector cells comprising antiviral antibodies in an effective amount to treat the viral disease. In some embodiments, the effector cells comprising antiviral antibodies are administered with a further antiviral treatment, either sequentially or simultaneously.

As used herein, the term “treating” or “treatment” refers to the application or administration of a composition including one or more active agents to a subject, who has a target disease or disorder (e.g., infectious disease) or a symptom of the disease/disorder with the purpose to prevent, cure, heal, alleviate, relieve, alter, remedy, ameliorate, improve, or affect the disorder or the symptom of the disease.

Alleviating a target disease/disorder (e.g., infectious disease) includes delaying the development or progression of the disease, or reducing disease severity or prolonging survival. Alleviating the disease or prolonging survival does not necessarily require curative results. As used therein, “delaying” the development of a target disease or disorder means to defer, hinder, slow, retard, stabilize, and/or postpone progression of the disease. This delay can be of varying lengths of time, depending on the history of the disease and/or individuals being treated. A method that “delays” or alleviates the development of a disease, or delays the onset of the disease, is a method that reduces probability of developing one or more symptoms of the disease in a given time frame and/or reduces extent of the symptoms in a given time frame, when compared to not using the method. Such comparisons are typically based on clinical studies, using a number of subjects sufficient to give a statistically significant result.

“Development” or “progression” of a disease means initial manifestations and/or ensuing progression of the disease. Development of the disease can be detectable and assessed using standard clinical techniques as well known in the art. However, development also refers to progression that may be undetectable. For purpose of this disclosure, development or progression refers to the biological course of the symptoms. “Development” includes occurrence, recurrence, and onset. As used herein “onset” or “occurrence” of a target disease or disorder includes initial onset and/or recurrence.

Infectious diseases are those caused by microorganisms (e.g., viruses, bacteria, fungi, or parasites).

Viral diseases include but are not limited to those caused by Poxyiridae, Herpesviridae, Adenoviridae, Papillomaviridae, Polyomaviridae, Parvoviridae, Hepadnaviridae, Retroviridae, Reoviridae, Filoviridae, Paramyxoviridae, Rhabdoviridae, Orthomyxoviridae, Bunyaviridae, Arenaviridae, Coronaviridae, Picornaviridae, Hepeviridae, Caliciviridae, Astroviridae, Togaviridae, Flaviviridae, Deltavirus, Bornaviridae, and prions. Examples of viral diseases include, but are not limited to, herpes virus, arena virus, corona virus, enterovirus, fieldvirus, filovirus, flavivirus, hantavirus, rotavirus, arbovirus, Epstein-Barr virus, cytomegalovirus, infant cytomegalovirus, astroviruses, adenoviruses and lentiviruses. Diseases related to viral infections (viral diseases) include infectious serial species (molluscum contagiosum), coronavirus, HTLV, HTLV-1, HIV/AIDS, human papilloma virus, herpes virus, herpes, genital herpes, viral dysentery, colds, flu, measles, rubella, Chickenpox, mumps, gray myelitis, rabies, mononucleosis, ebola, respiratory syncytial virus (RSV), dengue, yellow fever, Lassa fever, viral meningitis, western Nile fever, parainfluenza, chickenpox, smallpox, dengue hemorrhagic fever, progressive multiple Progressive multifocal leukoencephalopathy, viral gastroenteritis, acute appendicitis, hepatitis A, hepatitis B, chronic hepatitis B, hepatitis C, chronic hepatitis C, hepatitis D, hepatitis E, hepatitis X, simple Herpes, shingles, meningitis, encephalitis, shingles, pneumonia, encephalitis, California serogroup viral, St. Louis encephalitis, Rift Valley fever, hand, foot & mouth disease, hendra virus, Japanese encephalitis, lymphocytic choroiditis, sudden rash, dorsal parasite virus, SARS, warts, and cerebellar disease, In one embodiment, the viral disease is caused by Coronaviridae and is SARS-COV-2.

Bacterial diseases may be caused by, for example, Acetobacter, Acinetobacter, Actinomyces, Agrobacterium, Anaplasma, Azorhizobia, Bacillus, Bacteroides, Bartonella, Bordetella, Borrelia, Brucella, Burkkolderia, Calymmatobacterium, Campylobacter, Chlamydia, Chlamydophila, Clostridium, Corynebacterium, Coxiella, Ehrlichia, Enterobacter, Enterococcus, Escherichia, Francisella, Fusobacterium, Gardnerella, Haemophilus, Helicobacter, Klebsiella, Lactobacillus, Legionella, Listeria, Methanobacterium, Microbacterium, Micrococcus, Moraxella, Mycobacterium, Mycoplasma, Neisseria, Pasteurella, Peptostreptococcus, Porphyromonas, Prevotella, Pseudomonas, Rhizobium, Rickettsia, Rochalimaea, Rothia, Salmonella, Shigella, Staphylococcus, Stenotrophomonas, Streptococcus, Treponema, Vibrio, Walbachia, and Yersinia. In one embodiment, the bacterial infection is a Streptococcus pneumoniae infection.

Examples of fungal infections include, but are not limited to, aspergillosis, blastomycosis, candidiasis, coccidioidomycosis (Valley Fever), cryptococcosis, histoplasmosis, mucormycosis, Pneumocystis pneumonia (PCP), ringworm, sporotrichosis, and talaromycosis, In some embodiments, the fungal disease is caused by a Cryptococcus, Aspergillus, Candida, Coccidioides, Blastomyces, Ajellomyces, Histoplasma, Rhizopus, Apophysomyces, Absidia, Saksenaea, Rhizomucor pusillus, Entomophthora, Conidiobolus, Basidiobolus, Sporothrix,Pneumocystis jirovecii, Talaromyces marneffei, Asclepias, Fusarium, or Scedosporium fungus/species. In some embodiments, the fungal disease is caused by a fungal species including, but not limited to, Aspergillus fumigatus, Aspergillus flavus, Aspergillus niger, Aspergillus terreus, Blastomyces dermatitidis, Ajellomyces dermatitidis, Candida albicans, Candida auris, Candida glabrata, Candida parapsilosis, Candida rugosa, Candida tropicalis, Coccidioides immitis, Coccidioides posadasii, Cryptococcus neoformans, Cryptococcus gattii, Histoplasma capsulatum, Rhizopus stolonifer, Rhizopus arrhizus, Mucor indicus, Cunninghamella bertholletiae, Apophysomyces elegans, Absidia species, Saksenaea species, Rhizomucor pusillus, Entomophthora species, Conidiobolus species, Basidiobolus species, Sporothrix schenckii, Pneumocystis jirovecii, Talaromyces marneffei, Asclepias albicans, Fusarium solani, Scedosporium apiospermum, and Rhizomucor pusillus.

Examples of parasitic infections include, but are not limited to, African trypanosomiasis, Amebiasis, Ascariasis, Babesiosis, Chagas Disease, Clonorchiasis, Cryptosporidiosis, Cysticercosis, Diphyllobothriasis, Dracunculiasis, Echinococcosis, Enterobiasis, Fascioliasis, Fasciolopsiasis, Filariasis, Free-living amebic infection, Giardiasis, Gnathostomiasis, Hymenolepiasis, Isosporiasis, Kala-azar, Leishmaniasis, Malaria, Metagonimiasis, Myiasis, Onchocerciasis, Pediculosis, Pinworm Infection, Scabies, Schistosomiasis, Taeniasis, Toxocariasis, Toxoplasmosis, Trichinellosis, Trichinosis, Trichuriasis, and Trypanosomiasis.

Conventional methods, known to those of ordinary skill in the art of medicine, can be used to administer the pharmaceutical composition to the subject, depending upon the type of disease to be treated or the site of the disease. This composition can also be administered via other conventional routes, e.g., administered orally, parenterally, by inhalation spray, topically, rectally, nasally, buccally, vaginally or via an implanted reservoir. The term “parenteral” as used herein includes subcutaneous, intracutaneous, intravenous, intramuscular, intraarticular, intraarterial, intrasynovial, intrasternal, intrathecal, intralesional, and intracranial injection or infusion techniques. In one embodiment, the composition is administered via intratumoral injection. In a specific embodiment, the composition is administered intravenously. In addition, it can be administered to the subject via injectable depot routes of administration such as using 1-, 3-, or 6-month depot injectable or biodegradable materials and methods. In some examples, the pharmaceutical composition is administered intraocularly or intravitreally.

Injectable compositions may contain various carriers such as vegetable oils, dimethylactamide, dimethyformamide, ethyl lactate, ethyl carbonate, isopropyl myristate, ethanol, and polyols (glycerol, propylene glycol, liquid polyethylene glycol, and the like). For intravenous injection, water soluble antibodies can be administered by the drip method, whereby a pharmaceutical formulation containing the antiviral cocktail or effector cells comprising antiviral antibodies and at least one immune checkpoint inhibitor and a physiologically acceptable excipient is infused. Physiologically acceptable excipients may include, for example, 5% dextrose, 0.9% saline, Ringer's solution or other suitable excipients. Intramuscular preparations, e.g., a sterile formulation of a suitable soluble salt form of the antiviral antibody, antiviral antibody cocktail, or effector cells comprising antiviral antibodies, can be dissolved and administered in a pharmaceutical excipient such as Water-for-Injection, 0.9% saline, or 5% glucose solution.

In one embodiment, a composition is administered via site-specific or targeted local delivery techniques. Examples of site-specific or targeted local delivery techniques include various implantable depot sources of the composition or local delivery catheters, such as infusion catheters, an indwelling catheter, or a needle catheter, synthetic grafts, adventitial wraps, shunts and stents or other implantable devices, site specific carriers, direct injection, or direct application. See, e.g., PCT Publication No. WO 00/53211 and U.S. Pat. No. 5,981,568.

The particular dosage regimen, i.e., dose, timing, and repetition, used in the method described herein will depend on the particular subject and that subject's medical history. In some embodiments, the armed effector cells are administered to the subject at least 1, 2, 3, 4, 5, 6, 7, 8, 9, 10, 11, 12, 13, 14, 15, 16, 17, 18, 19, 20, or more times. In one embodiment, the armed effector cells are administered to the subject at least twice. In another embodiment, the armed effector cells are administered to the subject at least three times.

In some embodiments, the armed effector cells produce a significant reduction in symptoms, for example, relative to controls treated with PBS or an isotype antibody. In some embodiments, the armed effector cells produce a significant reduction in infectious disease severity (e.g., viral load). In some embodiments, the armed effector cells produce a significant increase in survival rate, for example, relative to controls treated with PBS or an isotype antibody.

In some embodiments, the armed effector cells produce a durable immune response; that is, when the subject is exposed to the antigen (e.g., infectious disease antigen) one or more subsequent times, the subject mounts an effective anti-infectious agent immune response. In some embodiments, the durable immune response persists for 1 day, 2 days, 3 days, 4 days, 5 days, 6 days, 1 week, 1.5 weeks, 2 weeks, 2.5 weeks, 3 weeks, 3.5 weeks, 1 month, 2 months, 3 months, 4 months, 5 months, 6 months, 7 months, 8 months, 9 months, 10 months, 11 months, 1 year, 1.5 years, 2 years, or longer, following an initial exposure to the antigen (e.g., infectious disease antigen) and treatment with any of the compositions described herein. In one embodiment, the durable immune response persists for at least six months.

In some embodiments, more than one composition, or a combination of a compositions described herein and another suitable therapeutic agent, may be administered to a subject in need of the treatment. The composition described herein can also be used in conjunction with other agents that serve to enhance and/or complement the effectiveness of the agents.

Thus, in one embodiment, the methods of the disclosure can be used in conjunction with one or more infectious disease therapeutics, for example, in conjunction with an antiviral agent, an antibacterial agent, an anti-fungal agent, an anti-parasitic agent or a traditional vaccine (e.g., viral vaccine) (e.g., simultaneously, or as part of an overall treatment procedure). Parameters of infectious disease treatment that may vary include, but are not limited to, dosages, timing of administration or duration or therapy; and the infectious disease treatment can vary in dosage, timing, or duration. Any agent or therapy (e.g., antiviral agents, antibacterial agents, anti-fungal agents, anti-parasitic agents) which is known to be useful, or which has been used or is currently being used for the prevention or treatment of the infectious disease can be used in combination with a composition of the disclosure in accordance with the disclosure described herein. One of ordinary skill in the medical arts can determine an appropriate treatment for a subject.

Examples of antibacterial agents include, but are not limited to, macrolides and ketolides (erythromycin, azithromycin, clarithromycin and telithromycin), beta-lactams (penicillin, cephalosporin and carbapenem drugs such as carbapenem, imipenem and meropenem), monobactams (penicillin G, penicillin V, methicillin, oxacillin, cloxacillin, dicloxacillin, nafcillin, ampicillin, amoxicillin, carbenicillin, ticarcillin, mezlocillin, piperacillin, azlocillin, temocillin, cepalotina, cephapirin, cephradine, cephaloridine, cefazolin, cefamandole, cefuroxime, cephalexin, cefprozil, cefaclor, loracarbef, cefoxitin, cefmetazol, cefotaxime, ceftizoxime, ceftriaxone, cefoperazone, ceftazidime, cefixime, cefpodoxime, ceftibuten, cefdinir, cefpiroma, cefepime and astreonam), quinolones (nalidixic acid, oxolinic acid, norfloxacin, pefloxacin, enoxacin, ofloxacin, levofloxacin, ciprofloxacin, temafloxacin, lomefloxacin, fleroxacin, grepafloxacin, sparfloxacin, trovafloxacin, clinafloxacin, gatifloxacin, moxifloxacin, sitafloxacin, ganefloxacina, gemifloxacin and pazufloxacin), antibacterial sulfonamides and antibacterial sulfanilamides (para-aminobenzoic acid, sulfadiazine, sulfisoxazole, sulfamethoxazole and sulfatalidine), aminoglycosides (streptomycin, neomycin, kanamycin, paromycin, gentamicin, tobramycin, amikacin, netinicin, spectinomycin, sisomycin, dibekacin and isepamycin), tetracyclines (tetracycline, chlortetracycline, demeclocycline, minocycline, oxytetracycline, methacycline, doxycycline), rifamycins (rifampin (also called rifampin), rifapentin, rifabutin, bezoxazinorifamycin and rifaximin), lincosamides (lincomycin and clindamycin), glycopeptides (vancomycin and teicoplanin), streptogramins (quinupristin and daflopristine), oxazolidinones (linezolid), polymyxin, colistin, colimycin, trimethoprim, and bacitracin and derivatives and analogs thereof.

Examples of antiviral agents include, but are not limited to, Abacavir, Acyclovir (Aciclovir), Adefovir, Amantadine, Ampligen, Amprenavir (Agenerase), Arbidol, Atazanavir, Atripla, Balavir, Baloxavir marboxil (Xofluza), Biktarvy, Boceprevir (Victrelis), Cidofovir, Cobicistat (Tybost), Combivir, Daclatasvir (Daklinza), Darunavir, Delavirdine, Descovy, Didanosine, Docosanol, Dolutegravir, Doravirine (Pifeltro), Ecoliever, Edoxudine, Efavirenz, Elvitegravir, Emtricitabine, Enfuvirtide, Entecavir, Etravirine (Intelence), Famciclovir, Fomivirsen, Fosamprenavir, Foscarnet, Fosfonet, Fusion inhibitors, Ganciclovir (Cytovene), Ibacitabine, Ibalizumab (Trogarzo), Idoxuridine, Imiquimod, Imunovir, Indinavir, Inosine, Integrase inhibitor, Interferon type I, Interferon type II, Interferon type III, Lamivudine, Letermovir (Prevymis), Lopinavir, Loviride, Maraviroc, Methisazone, Moroxydine, Nelfinavir, Nevirapine, Nexavir, Nitazoxanide, Norvir, Nucleoside analogues, Oseltamivir (Tamiflu), Peginterferon alfa-2a, Peginterferon alfa-2b, Penciclovir, Peramivir (Rapivab), Pleconaril, Podophyllotoxin, Protease inhibitors, Pyramidine, Raltegravir, Remdesivir, Reverse transcriptase inhibitors, Ribavirin, Rilpivirine (Edurant), Rimantadine, Ritonavir, Saquinavir, Simeprevir (Olysio), Sofosbuvir, Stavudine, Telaprevir, Telbivudine (Tyzeka), Tenofovir alafenamide, Tenofovir disoproxil, Tenofovir, Tipranavir, Trifluridine, Trizivir, Tromantadine, Truvada, Valaciclovir (Valtrex), Valganciclovir, Vicriviroc, Vidarabine, Viramidine, Zalcitabine, Zanamivir (Relenza), and Zidovudine, and derivatives and analogs thereof.

Examples of anti-fungal agents include, but are not limited to, zoles (e.g., Fluconazole®, Itraconazole®, Ketoconazole®, Miconazole®, Clortrimazole®, Voriconazole®, Posaconazole®, Rovuconazole®, etc.), polyenes (e.g., natamycin, lucensomycin, nystatin, amphotericin B, etc.), echinocandins (e.g., Cancidas®), pradimicins (e.g., beanomicins, nikkomycins, sordarins, allylamines, etc.) and derivatives and analogs thereof.

Examples of additional agents for purposes of treating parasitic infections include, but are not limited to, antihelminthic agents (e.g., albendazole (Albenza), mebendazole (Vermox), niclosamide (Niclocide), oxamniquine (Vansil), praziquantel (Biltricide), pyrantel (Antiminth), pyantel pamoate (Antiminth), thiabendazole (Mintezol), bitional, ivermectin, and diethylcarbamazepine citrate and derivatives and analogs thereof.

In specific embodiments, an appropriate anti-infectious disease regimen is selected depending on the type of disease (e.g., by a physician). For instance, a patient with SARS-COV-2 may be administered a prophylactically or therapeutically effective amount of a composition comprising effector cells (e.g., WBCs) armed with antiviral antibodies

Provided herein, in some aspects is a composition comprising armed effector cells and a pharmaceutically acceptable carrier (excipient). “Acceptable” means that the carrier must be compatible with the active ingredient of the composition (and preferably, capable of stabilizing the active ingredient) and not deleterious to the subject to be treated. Pharmaceutically acceptable excipients (carriers) including buffers, which are well known in the art. See, e.g., Remington: The Science and Practice of Pharmacy 20th Ed. (2000) Lippincott Williams and Wilkins, Ed. K. E. Hoover.

The pharmaceutical compositions to be used in the present methods can comprise pharmaceutically acceptable carriers, excipients, or stabilizers in the form of lyophilized formulations or aqueous solutions. (Remington: The Science and Practice of Pharmacy 20th Ed. (2000) Lippincott Williams and Wilkins, Ed. K. E. Hoover). Acceptable carriers, excipients, or stabilizers are nontoxic to recipients at the dosages and concentrations used, and may comprise buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrans; chelating agents such as EDTA; sugars such as sucrose, mannitol, trehalose or sorbitol; salt-forming counter-ions such as sodium; metal complexes (e.g. Zn-protein complexes); and/or non-ionic surfactants such as TWEEN™, PLURONICS™ or polyethylene glycol (PEG).

In some examples, the pharmaceutical composition described herein comprises liposomes containing the antibodies which can be prepared by methods known in the art, such as described in Epstein, et al., Proc. Natl. Acad. Sci. USA 82:3688 (1985); Hwang, et al., Proc. Natl. Acad. Sci. USA 77:4030 (1980); and U.S. Pat. Nos. 4,485,045 and 4,544,545. Liposomes with enhanced circulation time are disclosed in U.S. Pat. No. 5,013,556. Particularly useful liposomes can be generated by the reverse phase evaporation method with a lipid composition comprising phosphatidylcholine, cholesterol and PEG-derivatized phosphatidylethanolamine (PEG-PE). Liposomes are extruded through filters of defined pore size to yield liposomes with the desired diameter.

The antibodies may also be entrapped in microcapsules prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nano-particles and nanocapsules) or in macroemulsions. Such techniques are known in the art, see, e.g., Remington, The Science and Practice of Pharmacy 20th Ed. Mack Publishing (2000).

In other examples, the pharmaceutical composition described herein can be formulated in sustained-release format. Suitable examples of sustained-release preparations include semipermeable matrices of solid hydrophobic polymers containing the antibody, which matrices are in the form of shaped articles, e.g. films, or microcapsules. Examples of sustained-release matrices include polyesters, hydrogels (for example, poly(2-hydroxyethyl-methacrylate), or poly(vinyl alcohol)), polylactides (U.S. Pat. No. 3,773,919), copolymers of L-glutamic acid and 7 ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate), sucrose acetate isobutyrate, and poly-D-(−)-3-hydroxybutyric acid.

The pharmaceutical compositions to be used for in vivo administration must be sterile. This is readily accomplished by, for example, filtration through sterile filtration membranes. Therapeutic antibody compositions are generally placed into a container having a sterile access port, for example, an intravenous solution bag or vial having a stopper pierceable by a hypodermic injection needle.

The pharmaceutical compositions described herein can be in unit dosage forms such as tablets, pills, capsules, powders, granules, solutions or suspensions, or suppositories, for oral, parenteral or rectal administration, or administration by inhalation or insufflation.

For preparing solid compositions such as tablets, the principal active ingredient can be mixed with a pharmaceutical carrier, e.g., conventional tableting ingredients such as corn starch, lactose, sucrose, sorbitol, talc, stearic acid, magnesium stearate, dicalcium phosphate or gums, and other pharmaceutical diluents, e.g., water, to form a solid preformulation composition containing a homogeneous mixture of a compound of the present invention, or a non-toxic pharmaceutically acceptable salt thereof. When referring to these preformulation compositions as homogeneous, it is meant that the active ingredient is dispersed evenly throughout the composition so that the composition may be readily subdivided into equally effective unit dosage forms such as tablets, pills and capsules. This solid preformulation composition is then subdivided into unit dosage forms of the type described above containing from 0.1 to about 500 mg of the active ingredient of the present invention. The tablets or pills of the novel composition can be coated or otherwise compounded to provide a dosage form affording the advantage of prolonged action. For example, the tablet or pill can comprise an inner dosage and an outer dosage component, the latter being in the form of an envelope over the former. The two components can be separated by an enteric layer that serves to resist disintegration in the stomach and permits the inner component to pass intact into the duodenum or to be delayed in release. A variety of materials can be used for such enteric layers or coatings, such materials including a number of polymeric acids and mixtures of polymeric acids with such materials as shellac, cetyl alcohol and cellulose acetate.

Suitable surface-active agents include, in particular, non-ionic agents, such as polyoxyethylenesorbitans (e.g., Tween™ 20, 40, 60, 80 or 85) and other sorbitans (e.g., Span™ 20, 40, 60, 80 or 85). Compositions with a surface-active agent will conveniently comprise between 0.05 and 5% surface-active agent, and can be between 0.1 and 2.5%. It will be appreciated that other ingredients may be added, for example mannitol or other pharmaceutically acceptable vehicles, if necessary.

Suitable emulsions may be prepared using commercially available fat emulsions, such as Intralipid™, Liposyn™, Infonutrol™, Lipofundin™ and Lipiphysan™. The active ingredient may be either dissolved in a pre-mixed emulsion composition or alternatively it may be dissolved in an oil (e.g., soybean oil, safflower oil, cottonseed oil, sesame oil, corn oil or almond oil) and an emulsion formed upon mixing with a phospholipid (e.g. egg phospholipids, soybean phospholipids or soybean lecithin) and water. It will be appreciated that other ingredients may be added, for example glycerol or glucose, to adjust the tonicity of the emulsion. Suitable emulsions will typically contain up to 20% oil, for example, between 5 and 20%. The fat emulsion can comprise fat droplets between 0.1 and 1.0.im, particularly 0.1 and 0.5.im, and have a pH in the range of 5.5 to 8.0.

The emulsion compositions can be those prepared by mixing an antibody with Intralipid™ or the components thereof (soybean oil, egg phospholipids, glycerol and water).

Pharmaceutical compositions for inhalation or insufflation include solutions and suspensions in pharmaceutically acceptable, aqueous or organic solvents, or mixtures thereof, and powders. The liquid or solid compositions may contain suitable pharmaceutically acceptable excipients as set out above. In some embodiments, the compositions are administered by the oral or nasal respiratory route for local or systemic effect.

Compositions in preferably sterile pharmaceutically acceptable solvents may be nebulized by use of gases. Nebulized solutions may be breathed directly from the nebulizing device or the nebulizing device may be attached to a face mask, tent or intermittent positive pressure breathing machine. Solution, suspension or powder compositions may be administered, preferably orally or nasally, from devices which deliver the formulation in an appropriate manner.

To practice the method disclosed herein, an effective amount of the pharmaceutical composition described herein can be administered to a subject (e.g., a human) in need of the treatment via a suitable route, such as intravenous administration, e.g., as a bolus or by continuous infusion over a period of time, by intramuscular, intraperitoneal, intracerebrospinal, subcutaneous, intra-articular, intrasynovial, intrathecal, oral, inhalation or topical routes. Commercially available nebulizers for liquid formulations, including jet nebulizers and ultrasonic nebulizers are useful for administration. Liquid formulations can be directly nebulized and lyophilized powder can be nebulized after reconstitution. Alternatively, the antibodies as described herein can be aerosolized using a fluorocarbon formulation and a metered dose inhaler, or inhaled as a lyophilized and milled powder.

The subject to be treated by the methods described herein can be a mammal, more preferably a human. Mammals include, but are not limited to, farm animals, sport animals, pets, primates, horses, dogs, cats, mice and rats. A human subject who needs the treatment may be a human patient having, at risk for, or suspected of having a target disease/disorder, such as an infectious disease.

A subject suspected of having any of such target disease/disorder (e.g., infectious disease) might show one or more symptoms of the disease/disorder. A subject at risk for the disease/disorder can be a subject having one or more of the risk factors for that disease/disorder.

As used herein, “an effective amount” refers to the amount of each active agent required to confer therapeutic effect on the subject, either alone or in combination with one or more other active agents. In some embodiments, the therapeutic effect is reduced infectious agent bioactivity. Determination of whether an amount of the antibody achieved the therapeutic effect would be evident to one of skill in the art. Effective amounts vary, as recognized by those skilled in the art, depending on the particular condition being treated, the severity of the condition, the individual patient parameters including age, physical condition, size, gender and weight, the duration of the treatment, the nature of concurrent therapy (if any), the specific route of administration and like factors within the knowledge and expertise of the health practitioner. These factors are well known to those of ordinary skill in the art and can be addressed with no more than routine experimentation. It is generally preferred that a maximum dose of the individual components or combinations thereof be used, that is, the highest safe dose according to sound medical judgment.

Empirical considerations, such as the half-life, generally will contribute to the determination of the dosage. For example, antibodies that are compatible with the human immune system, such as humanized antibodies or fully human antibodies, may be used to prolong half-life of the antibody and to prevent the antibody being attacked by the host's immune system. Frequency of administration may be determined and adjusted over the course of therapy, and is generally, but not necessarily, based on treatment and/or suppression and/or amelioration and/or delay of a target disease/disorder. Alternatively, sustained continuous release formulations of an antibody may be appropriate. Various formulations and devices for achieving sustained release are known in the art.

In one example, dosages for an antibody as described herein may be determined empirically in individuals who have been given one or more administration(s) of the antibody. Individuals are given incremental dosages of the antagonist. To assess efficacy of the antagonist, an indicator of the disease/disorder can be followed.

Generally, for administration of any of the antibodies described herein, an initial candidate dosage can be about 2 mg/kg. For the purpose of the present disclosure, a typical daily dosage might range from about any of 0.1 μg/kg to 3 μg/kg to 30 μg/kg to 300 μg/kg to 3 mg/kg, to 30 mg/kg to 100 mg/kg or more, depending on the factors mentioned above. In some embodiments, the dosage of antibody administered is measured as a volume of convalescent plasma. In some embodiments, the volume of convalescent plasma is 10 mL, 20 mL, 25 mL, 30 mL, 35 mL, 40 mL, 45, mL 50 mL, 55 mL, 60 mL, 65 mL, 70 mL, 75 mL, 80 mL, 85 mL, 90 mL, 95 mL, 100 mL or more. In one embodiment, the volume of convalescent plasma is 50 mL. For repeated administrations over several days or longer, depending on the condition, the treatment is sustained until a desired suppression of symptoms occurs or until sufficient therapeutic levels are achieved to alleviate a target disease or disorder, or a symptom thereof. An exemplary dosing regimen comprises administering an initial dose of about 50 mL, followed by daily maintenance doses of about 25-50 mL of the antibody. A further exemplary dosage regimen comprises administering an initial dose of 50 mL followed by daily maintenance of 50 mL, for a total of 2, 3, 4, 5, 6, 7, 8, 9, 10, or more doses/days. However, other dosage regimens may be useful, depending on the pattern of pharmacokinetic decay that the practitioner wishes to achieve. For example, dosing from 5-10 times a week is contemplated. The progress of this therapy is easily monitored by conventional techniques and assays. The dosing regimen (including the antibody, antibodies, or convalescent plasma used) can vary over time.

In some embodiments, for an adult patient of normal weight, doses ranging from about 0.3 to 5.00 mg/kg may be administered. In some examples, the dosage of the effector cells comprising antibodies described herein can be 10 mg/kg. The particular dosage regimen, i.e., dose, timing, and repetition, will depend on the particular individual and that individual's medical history, as well as the properties of the individual agents (such as the half-life of the agent, and other considerations well known in the art).

For the purpose of the present disclosure, the appropriate dosage of an antibody as described herein will depend on the specific antibody, antibodies, and/or non-antibody peptide (or compositions thereof) employed, the type and severity of the disease/disorder, whether the antibody is administered for preventive or therapeutic purposes, previous therapy, the patient's clinical history and response to the antagonist, and the discretion of the attending physician. Typically the clinician will administer an antibody, until a dosage is reached that achieves the desired result. In some embodiments, the desired result is an increase in anti-tumor immune response in the tumor microenvironment. Methods of determining whether a dosage resulted in the desired result would be evident to one of skill in the art. Administration of one or more antibodies can be continuous or intermittent, depending, for example, upon the recipient's physiological condition, whether the purpose of the administration is therapeutic or prophylactic, and other factors known to skilled practitioners. The administration of an antibody may be essentially continuous over a preselected period of time or may be in a series of spaced dose, e.g., either before, during, or after developing a target disease or disorder.

The present disclosure also provides kits for use in treating infectious diseases. Such kits can include one or more containers comprising effector cells comprising convalescent plasma (antibodies).

In some embodiments, the kit can comprise instructions for use in accordance with any of the methods described herein. The included instructions can comprise a description of administration of the effector cells comprising antibodies, and optionally the second therapeutic agent, to treat, delay the onset, or alleviate a target disease as those described herein. The kit may further comprise a description of selecting an individual suitable for treatment based on identifying whether that individual has the target disease, e.g., applying a diagnostic method as described herein. In still other embodiments, the instructions comprise a description of administering an antibody to an individual at risk of the target disease.

The instructions relating to the use of effector cells comprising antibodies generally include information as to dosage, dosing schedule, and route of administration for the intended treatment. The containers may be unit doses, bulk packages (e.g., multi-dose packages) or sub-unit doses. Instructions supplied in the kits of the invention are typically written instructions on a label or package insert (e.g., a paper sheet included in the kit), but machine-readable instructions (e.g., instructions carried on a magnetic or optical storage disk) are also acceptable.

The label or package insert indicates that the composition is used for treating or alleviating an infectious disease. Instructions may be provided for practicing any of the methods described herein.

The kits of this invention are in suitable packaging. Suitable packaging includes, but is not limited to, vials, bottles, jars, flexible packaging (e.g., sealed Mylar or plastic bags), and the like. Also contemplated are packages for use in combination with a specific device, such as an inhaler, nasal administration device (e.g., an atomizer) or an infusion device such as a minipump. A kit may have a sterile access port (for example the container may be an intravenous solution bag or a vial having a stopper pierceable by a hypodermic injection needle). The container may also have a sterile access port (for example the container may be an intravenous solution bag or a vial having a stopper pierceable by a hypodermic injection needle). At least one active agent in the composition is an effector cell comprising an antibody (e.g., from convalescent plasma), such as those described herein.

Kits may optionally provide additional components such as buffers and interpretive information. Normally, the kit comprises a container and a label or package insert(s) on or associated with the container. In some embodiments, the invention provides articles of manufacture comprising contents of the kits described above.

The practice of the present invention will employ, unless otherwise indicated, conventional techniques of molecular biology (including recombinant techniques), microbiology, cell biology, biochemistry and immunology, which are within the skill of the art. Such techniques are explained fully in the literature, such as, Molecular Cloning: A Laboratory Manual, second edition (Sambrook, et al., 1989) Cold Spring Harbor Press; Oligonucleotide Synthesis (M. J. Gait, ed., 1984); Methods in Molecular Biology, Humana Press; Cell Biology: A Laboratory Notebook (J. E. Cellis, ed., 1998) Academic Press; Animal Cell Culture (R. I. Freshney, ed., 1987); Introduction to Cell and Tissue Culture (J. P. Mather and P. E. Roberts, 1998) Plenum Press; Cell and Tissue Culture: Laboratory Procedures (A. Doyle, J. B. Griffiths, and D. G. Newell, eds., 1993-8) J. Wiley and Sons; Methods in Enzymology (Academic Press, Inc.); Handbook of Experimental Immunology (D. M. Weir and C. C. Blackwell, eds.); Gene Transfer Vectors for Mammalian Cells (J. M. Miller and M. P. Calos, eds., 1987); Current Protocols in Molecular Biology (F. M. Ausubel, et al., eds., 1987); PCR: The Polymerase Chain Reaction, (Mullis, et al., eds., 1994); Current Protocols in Immunology (J. E. Coligan et al., eds., 1991); Short Protocols in Molecular Biology (Wiley and Sons, 1999); Immunobiology (C. A. Janeway and P. Travers, 1997); Antibodies (P. Finch, 1997); Antibodies: a practical approach (D. Catty., ed., IRL Press, 1988-1989); Monoclonal antibodies: a practical approach (P. Shepherd and C. Dean, eds., Oxford University Press, 2000); Using antibodies: a laboratory manual (E. Harlow and D. Lane (Cold Spring Harbor Laboratory Press, 1999); The Antibodies (M. Zanetti and J. D. Capra, eds., Harwood Academic Publishers, 1995).

Without further elaboration, it is believed that one skilled in the art can, based on the above description, utilize the present invention to its fullest extent. The following specific embodiments are, therefore, to be construed as merely illustrative, and not limitative of the remainder of the disclosure in any way whatsoever. All publications cited herein are incorporated by reference for the purposes or subject matter referenced herein.

### EXAMPLES

In order that the invention described herein may be more fully understood, the following examples are set forth. The examples described in this application are offered to illustrate the compounds, pharmaceutical compositions, and methods provided herein and are not to be construed in any way as limiting their scope.

### Example 1. Treatment of COVID-19 Using Low Dose Antibodies to Redirect White Blood Cells

This is a non-randomized, open-label study in patients with active SARS-CoV-2 (COVID-19) to determine the safety and feasibility of treatment using low dose anti-SARS-COV-2 antibodies to arm and redirect white blood cells (WBCs). Treatments will be administered in an innovative manner that drastically lowers the per treatment dose of antibodies. This is done by arming WBCs derived from low volume leukapheresis with SARS-COV-2 antibodies. Individual patients may benefit by improved treatment outcomes. The study is performed to determine, in patients with active, symptomatic COVID-19 disease: the safety of autologous white blood cells for delivering low dose anti-SARS-COV-2 antibodies, the feasibility of arming WBCs in ill patients while under strict isolation, and the duration and range of total WBCs that can be armed with anti-SARS-COV-2 antibodies.

Patient inclusionary criteria include: 18 years or older, positive of SARS-COV-2 infection, hospitalized due to SARS-COV-2 infection, NEW score ≥3, and ABO-compatible convalescent plasma available. Patients who have a history of allergic reactions to blood or plasma products, who are unlikely to comply with the study requirements, and/or have conditions in which proposed treatments (e.g., leukapheresis) may be dangerous are excluded from the study.

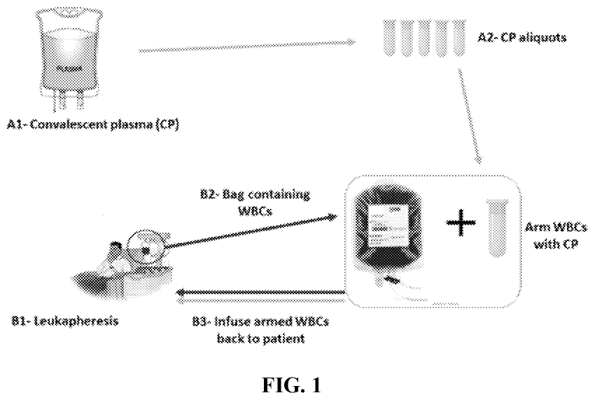

Ten patients are enrolled in the study, which is schematically illustrated in FIG. 1. Briefly, convalescent plasma (CP) from a donor that recovered from infection with SARS-COV-2 virus is processed and supplied by the blood bank according to FDA guidelines (A1). Next, the blood bank or pharmacy prepares aliquots of CP for each treatment (A2). WBCs are collected by leukapheresis from the patient with active COVID-19 disease (B1). The leukapheresis bag containing the WBCs is transferred to the blood bank laboratory (B2), where the aliquot of CP is added to the leukapheresis bag of WBCs and incubated for 30 minutes (“arming” step). Then, the armed WBCs are transported back to the patient and infused intravenously (B3).

This protocol constitutes a single treatment. Each treatment lasts less than 4 hours. Each treatment is designed to arm all of the WBCs available from and contained within the leukapheresis bag. The number of WBCs per treatment varies according to the patient's clinical status. Variables include the patients WBC count and the duration time of leukapheresis.

The final volume of WBCs armed with CP is 100 ml to 200 ml. Twenty ml of the armed WBCs is delivered over 20 minutes. If no transfusion reactions are observed the remainder of the armed WBCs is delivered over 30 to 60 minutes. The dose of antibody per treatment is described as volume of convalescent plasma. Each dose of CP is 50 mL per treatment. The cumulative dose is determined by the number of days of treatment. The duration of individual patient exposure is 3 days (patients will be treated with 3 consecutive daily treatments).

Assessments occur daily on study days 1-7, and on study day 14 and on study day 28. The following assessments are performed: total days of treatment, total volume of CP, total cumulative number of armed WBCs delivered, vital signs, including SpO2, assessment of clinical status (6-point ordinal scale), Glascow coma scale, NEW score, organ dysfunction using SOFA score, measures of clinical support (hospitalization, oxygen requirement, mechanical ventilator requirement, ICU requirement), chest x-ray finding (if performed for clinical indication), assessment of ARDS (PaO2 (or SpO2), FiO2, and recent chest x-ray data), CBC (differential white cell count (to include neutrophil and lymphocyte percentages), hemoglobin, hematocrit, and platelets), blood chemistries (creatinine, glucose, total protein, ALT/GPT, AST/GOT, total bilirubin), and prothrombin time and international normalized ratio (PT/INR). Adverse events, if any, are also noted.

Statistical description of the within subject and between subject data is based upon characterization of the numbers of WBCs, and their potential relationships to observed clinical side effects using exact methods for small sample sizes. In a similar fashion patient clinical and treatment features are examined to characterize potential differences as well as with duration of treatment. Specifically, within subject changes from baseline levels and intermediate observations to the final observed levels are characterized using a robust estimate of the slope over time based upon the median of all possible pairwise differences. Between subject comparisons are based upon patient groupings such as age categories, initial clinical status, and other patient characteristics. Most comparisons have small sample sizes and are examined using exact permutation methods.

### REFERENCES

1. Dye, J. M., A. S. Herbert, A. I. Kuehne, J. F. Barth, M. A. Muhammad, S. E. Zak, R. A. Ortiz, L. I. Prugar, and W. D. Pratt, Postexposure antibody prophylaxis protects nonhuman primates from filovirus disease. Proc Natl Acad Sci USA, 2012. 109(13): p. 5034-9.
2. Liu, M., R. L. Roberts, B. J. Ank, J. G. Marmet, and E. R. Stiehm, Antibody-directed natural cytotoxicity results in enhanced killing of HIV gp120-coated CEMNKR cells. Clin Immunol Immunopathol, 1997. 83(2): p. 139-46.
3. Balachandran, N., S. Bacchetti, and W. E. Rawls, Protection against lethal challenge of BALB/c mice by passive transfer of monoclonal antibodies to five glycoproteins of herpes simplex virus type 2. Infect Immun, 1982. 37(3): p. 1132-7.
4. Cheng, Y., R. Wong, Y. O. Soo, W. S. Wong, C. K. Lee, M. H. Ng, P. Chan, K. C. Wong, C. B. Leung, and G. Cheng, Use of convalescent plasma therapy in SARS patients in Hong Kong. Eur J Clin Microbiol Infect Dis, 2005. 24(1): p. 44-6.
5. Wong, V. W., D. Dai, A. K. Wu, and J. J. Sung, Treatment of severe acute respiratory syndrome with convalescent plasma. Hong Kong Med J, 2003. 9(3): p. 199-201.
6. Vittecoq, D., S. Chevret, L. Morand-Joubert, F. Heshmati, F. Audat, M. Bary, T. Dusautoir, A. Bismuth, J. P. Viard, F. Barre-Sinoussi, and et al., Passive immunotherapy in AIDS: a double-blind randomized study based on transfusions of plasma rich in anti-human immunodeficiency virus 1 antibodies vs. transfusions of seronegative plasma. Proc Natl Acad Sci USA, 1995. 92(4): p. 1195-9.
7. Blick, G., W. F. Scott, S. W. Crook, S. Buchanan, T. Garton, U. Hopkins, A. M. Vadaboncoeur, J. Doolittle, I. A. Bulcraig, P. Greiger-Zanlungo, and A. Karpas, Passive immunotherapy in advanced HIV infection and therapeutic plasmapheresis in asymptomatic HIV-positive individuals: a four-year clinical experience. Biotherapy, 1998. 11(1): p. 7-14.
8. Ko, J. H., H. Seok, S. Y. Cho, Y. E. Ha, J. Y. Baek, S. H. Kim, Y. J. Kim, J. K. Park, C. R. Chung, E. S. Kang, D. Cho, M. A. Muller, C. Drosten, C. I. Kang, D. R. Chung, J. H. Song, and K. R. Peck, Challenges of convalescent plasma infusion therapy in Middle East respiratory coronavirus infection: a single centre experience. Antivir Ther, 2018. 23(7): p. 617-622.
9. Luke, T. C., E. M. Kilbane, J. L. Jackson, and S. L. Hoffman, Meta-analysis: convalescent blood products for Spanish influenza pneumonia: a future H5N1 treatment? Ann Intern Med, 2006. 145(8): p. 599-609.
10. Kong, L. K. and B. P. Zhou, Successful treatment of avian influenza with convalescent plasma. Hong Kong Med J, 2006. 12(6): p. 489.
11. Zhou, B., N. Zhong, and Y. Guan, Treatment with convalescent plasma for influenza A (H5N1) infection. N Engl J Med, 2007. 357(14): p. 1450-1.
12. Shen, C., Z. Wang, F. Zhao, Y. Yang, J. Li, J. Yuan, F. Wang, D. Li, M. Yang, L. Xing, J. Wei, H. Xiao, Y. Yang, J. Qu, L. Qing, L. Chen, Z. Xu, L. Peng, Y. Li, H. Zheng, F. Chen, K. Huang, Y. Jiang, D. Liu, Z. Zhang, Y. Liu, and L. Liu, Treatment of 5 Critically Ill Patients With COVID-19 With Convalescent Plasma. JAMA, 2020.
13. Duan, K., B. Liu, C. Li, H. Zhang, T. Yu, J. Qu, M. Zhou, L. Chen, S. Meng, Y. Hu, C. Peng, M. Yuan, J. Huang, Z. Wang, J. Yu, X. Gao, D. Wang, X. Yu, L. Li, J. Zhang, X. Wu, B. Li, Y. Xu, W. Chen, Y. Peng, Y. Hu, L. Lin, X. Liu, S. Huang, Z. Zhou, L. Zhang, Y. Wang, Z. Zhang, K. Deng, Z. Xia, Q. Gong, W. Zhang, X. Zheng, Y. Liu, H. Yang, D. Zhou, D. Yu, J. Hou, Z. Shi, S. Chen, Z. Chen, X. Zhang, and X. Yang, Effectiveness of convalescent plasma therapy in severe COVID-19 patients. Proc Natl Acad Sci USA, 2020.
14. van Griensven, J., T. Edwards, X. de Lamballerie, M. G. Semple, P. Gallian, S. Baize, P. W. Horby, H. Raoul, N. Magassouba, A. Antierens, C. Lomas, O. Faye, A. A. Sall, K. Fransen, J. Buyze, R. Ravinetto, P. Tiberghien, Y. Claeys, M. De Crop, L. Lynen, E. I. Bah, P. G. Smith, A. Delamou, A. De Weggheleire, N. Haba, and C. Ebola-Tx, Evaluation of Convalescent Plasma for Ebola Virus Disease in Guinea. N Engl J Med, 2016. 374(1): p. 33-42.
15. van Griensven, J., T. Edwards, S. Baize, and C. Ebola-Tx, Efficacy of Convalescent Plasma in Relation to Dose of Ebola Virus Antibodies. N Engl J Med, 2016. 375(23): p. 2307-2309.
16. Davey, R. T., Jr., E. Fernandez-Cruz, N. Markowitz, S. Pett, A. G. Babiker, D. Wentworth, S. Khurana, N. Engen, F. Gordin, M. K. Jain, V. Kan, M. N. Polizzotto, P. Riska, K. Ruxrungtham, Z. Temesgen, J. Lundgren, J. H. Beigel, H. C. Lane, J. D. Neaton, and I.F.-I.S. Group, Anti-influenza hyperimmune intravenous immunoglobulin for adults with influenza A or B infection (FLU-IVIG): a double-blind, randomised, placebo-controlled trial. Lancet Respir Med, 2019. 7(11): p. 951-963.
17. Beigel, J. H., E. Aga, M. C. Elie-Turenne, J. Cho, P. Tebas, C. L. Clark, J. P. Metcalf, C. Ozment, K. Raviprakash, J. Beeler, H. P. Holley, Jr., S. Warner, C. Chorley, H. C. Lane, M. D. Hughes, R. T. Davey, Jr., and I. R. C. S. Team, Anti-influenza immune plasma for the treatment of patients with severe influenza A: a randomised, double-blind, phase 3 trial. Lancet Respir Med, 2019. 7(11): p. 941-950.
18. Wan, Y., J. Shang, S. Sun, W. Tai, J. Chen, Q. Geng, L. He, Y. Chen, J. Wu, Z. Shi, Y. Zhou, L. Du, and F. Li, Molecular Mechanism for Antibody-Dependent Enhancement of Coronavirus Entry. J Virol, 2020. 94(5).
19. Asmal, M., Y. Sun, S. Lane, W. Yeh, S. D. Schmidt, J. R. Mascola, and N. L. Letvin, Antibody-dependent cell-mediated viral inhibition emerges after simian immunodeficiency virus SIVmac251 infection of rhesus monkeys coincident with gp140-binding antibodies and is effective against neutralization-resistant viruses. J Virol, 2011. 85(11): p. 5465-75.
20. Rentier, B. and W. C. Wallen, Scanning and transmission electron microscopy study of antibody-dependent lymphocyte-mediated cytotoxicity on measles virus-infected cells. Infect Immun, 1980. 30(1): p. 303-15.
21. Zhong, X., Z. Guo, H. Yang, L. Peng, Y. Xie, T. Y. Wong, S. T. Lai, and Z. Guo, Amino terminus of the SARS coronavirus protein 3a elicits strong, potentially protective humoral responses in infected patients. J Gen Virol, 2006. 87(Pt 2): p. 369-73.
22. Brown, F. and C. J. Smale, Demonstration of three specific sites on the surface of foot-and-mouth disease virus by antibody complexing. J Gen Virol, 1970. 7(2): p. 115-27.
23. Hooghe-Peters, E. L., B. Rentier, and M. Dubois-Dalcq, Electron microscopic study of measles virus infection: unusual antibody-triggered redistribution of antigens on giant cells. J Virol, 1979. 29(2): p. 666-76.
24. Virelizier, J. L., A. C. Allison, J. S. Oxford, and G. C. Schild, Early presence of ribonucleoprotein antigen on surface of influenza virus-infected cells. Nature, 1977. 266(5597): p. 52-4.
25. Lustig, H. J. and C. Bianco, Antibody-mediated cell cytotoxicity in a defined system: regulation by antigen, antibody, and complement. J Immunol, 1976. 116(1): p. 253-60.
26. Yang, F., Y. Xiao, R. Lu, B. Chen, F. Liu, L. Wang, H. Yao, N. Wu, and H. Wu, Generation of neutralizing and non-neutralizing monoclonal antibodies against H7N9 influenza virus. Emerg Microbes Infect, 2020. 9(1): p. 664-675.
27. Amanat, F., P. Meade, S. Strohmeier, and F. Krammer, Cross-reactive antibodies binding to H4 hemagglutinin protect against a lethal H4N6 influenza virus challenge in the mouse model. Emerg Microbes Infect, 2019. 8(1): p. 155-168.
28. Rector, J. T., R. N. Lausch, and J. E. Oakes, Identification of infected cell-specific monoclonal antibodies and their role in host resistance to ocular herpes simplex virus type 1 infection. J Gen Virol, 1984. 65 (Pt 3): p. 657-61.
29. Wylie, D. E. and N. R. Klinman, The murine B cell repertoire responsive to an influenza-infected syngeneic cell line. J Immunol, 1981. 127(1): p. 194-8.
30. Vanderven, H. A., L. Liu, F. Ana-Sosa-Batiz, T. H. Nguyen, Y. Wan, B. Wines, P. M. Hogarth, D. Tilmanis, A. Reynaldi, M. S. Parsons, A. C. Hurt, M. P. Davenport, T. Kotsimbos, A. C. Cheng, K. Kedzierska, X. Zhang, J. Xu, and S. J. Kent, Fc functional antibodies in humans with severe H7N9 and seasonal influenza. JCI Insight, 2017. 2(13).
31. Kohl, S., N. C. Strynadka, R. S. Hodges, and L. Pereira, Analysis of the role of antibody-dependent cellular cytotoxic antibody activity in murine neonatal herpes simplex virus infection with antibodies to synthetic peptides of glycoprotein D and monoclonal antibodies to glycoprotein B. J Clin Invest, 1990. 86(1): p. 273-8.
32. Parrillo, J. E. and A. S. Fauci, Apparent direct cellular cytotoxicity mediated via cytophilic antibody. Multiple Fc receptor bearing effector cell populations mediating cytophilic antibody induced cytotoxicity. Immunology, 1977. 33(6): p. 839-50.
33. Penfold, P. L., L. C. Walker, and I. M. Roitt, Complex arming in antibody-dependent cell-mediated cytotoxicity: ultrastructural studies of the interaction between human effector cells armed with aggregated anti-DNP antibody and DNP-coated erythrocytes. Clin Exp Immunol, 1978. 31(2): p. 197-204.
34. Kohl, S., L. S. Loo, and L. K. Pickering, Protection of neonatal mice against herpes simplex viral infection by human antibody and leukocytes from adult, but not neonatal humans. J Immunol, 1981. 127(4): p. 1273-5.
35. Tyler, D. S., C. L. Nastala, S. D. Stanley, T. J. Matthews, H. K. Lyerly, D. P. Bolognesi, and K. J. Weinhold, GP120 specific cellular cytotoxicity in HIV-1 seropositive individuals. Evidence for circulating CD16+ effector cells armed in vivo with cytophilic antibody. J Immunol, 1989. 142(4): p. 1177-82.
36. Tyler, D. S., S. D. Stanley, C. A. Nastala, A. A. Austin, J. A. Bartlett, K. C. Stine, H. K. Lyerly, D. P. Bolognesi, and K. J. Weinhold, Alterations in antibody-dependent cellular cytotoxicity during the course of HIV-1 infection. Humoral and cellular defects. J Immunol, 1990. 144(9): p. 3375-84.
37. Shukla, G. S., S. C. Pero, Y. Sun, L. Mei, F. Zhang, G. Sholler, and D. N. Krag, Multiple antibodies targeting tumor-specific mutations redirect immune cells to inhibit tumor growth and increase survival in experimental animal models. Clin Transl Oncol, 2019: p. November 16.
38. Shukla, G. S., Y. J. Sun, S. C. Pero, and D. N. Krag, A cocktail of polyclonal affinity enriched antibodies against melanoma mutations increases binding and inhibits tumor growth. J Immunol Methods, 2020. 478: p. 112720.

### Other Embodiments

In the claims articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention includes embodiments in which more than one, or all of the group members are present in, employed in, or otherwise relevant to a given product or process.

Furthermore, the invention encompasses all variations, combinations, and permutations in which one or more limitations, elements, clauses, and descriptive terms from one or more of the listed claims is introduced into another claim. For example, any claim that is dependent on another claim can be modified to include one or more limitations found in any other claim that is dependent on the same base claim. Where elements are presented as lists, e.g., in Markush group format, each subgroup of the elements is also disclosed, and any element(s) can be removed from the group. It should it be understood that, in general, where the invention, or aspects of the invention, is/are referred to as comprising particular elements and/or features, certain embodiments of the invention or aspects of the invention consist, or consist essentially of, such elements and/or features. For purposes of simplicity, those embodiments have not been specifically set forth in haec verba herein. It is also noted that the terms “comprising” and “containing” are intended to be open and permits the inclusion of additional elements or steps. Where ranges are given, endpoints are included. Furthermore, unless otherwise indicated or otherwise evident from the context and understanding of one of ordinary skill in the art, values that are expressed as ranges can assume any specific value or sub-range within the stated ranges in different embodiments of the invention, to the tenth of the unit of the lower limit of the range, unless the context clearly dictates otherwise.

This application refers to various issued patents, published patent applications, journal articles, and other publications, all of which are incorporated herein by reference. If there is a conflict between any of the incorporated references and the instant specification, the specification shall control. In addition, any particular embodiment of the present invention that falls within the prior art may be explicitly excluded from any one or more of the claims. Because such embodiments are deemed to be known to one of ordinary skill in the art, they may be excluded even if the exclusion is not set forth explicitly herein. Any particular embodiment of the invention can be excluded from any claim, for any reason, whether or not related to the existence of prior art.

Those skilled in the art will recognize or be able to ascertain using no more than routine experimentation many equivalents to the specific embodiments described herein. The scope of the present embodiments described herein is not intended to be limited to the above Description, but rather is as set forth in the appended claims. Those of ordinary skill in the art will appreciate that various changes and modifications to this description may be made without departing from the spirit or scope of the present invention, as defined in the following claims.

1.-28. (canceled)

29. A method of treating an infectious disease in a subject, the method comprising:
(a) harvesting convalescent plasma from at least one donor;
(b) collecting effector cells from the subject, wherein the effector cells are selected from the group consisting of natural killer (NK) cells, neutrophils, T cells, B cells, monocytes/macrophages, and combinations thereof;
(c) incubating the convalescent plasma and effector cells together, thereby generating armed effector cells;
(d) administering the armed effector cells to the subject in an effective amount to treat the infectious disease.

30.-31. (canceled)

32. The method of claim 29, wherein the infectious disease is SARS-COV-2 (COVID19).

33. (canceled)

34. The method of claim 29, further comprising enriching the convalescent plasma.

35. The method of claim 29, wherein the effector cells are administered to the subject at least three times.

36. The method of claim 29, wherein the effector cells are administered daily.

37. The method of claim 29, wherein 50 mL of convalescent plasma is used to arm the effector cells.

38. (canceled)

39. The method of claim 29, wherein the effector cells are administered to the subject at least three times.

40. The method of claim 29, wherein the infectious disease is selected from the group consisting of: a viral disease, a bacterial disease, a fungal disease, and a parasitic disease.

41. The method of claim 39, wherein the infectious disease is a viral disease and the reduction in infectious disease severity is a reduction in viral load.

42. The method of claim 29, wherein the armed effector cells produce a significant reduction in infectious disease severity.


SEQUENCE LISTING (1 sequence)

SEQ ID NO: 1 - protein, 327 aa, Homo sapiens
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK